(19) United States
(12) Patent Application Publication
(10) Pub. No.: US 2019/0254992 A1
(43) Pub. Date: Aug. 22, 2019

(54) COMBINATIONS OF BETA-GLYCOLIPIDES AND 4-[(2-AMINO-3,5-DIBROMOPHENYL)METHYLAMINO]CYCLOHEXAN-1-OL, COMPOSITIONS AND USES THEREOF IN THE TREATMENT OF DISORDERS ASSOCIATED WITH PROTEIN MISFOLDING AND PROTEIN AGGREGATIONS

(71) Applicants: HADASIT MEDICAL RESEARCH SERVICES & DEVELOPMENT LIMITED, Jerusalem (IL); SHAARE ZEDEK SCIENTIFIC LTD., Jerusalem (IL)

(72) Inventors: David ARKADIR, Jerusalem (IL); Yaron ILAN, Kfar Tavor (IL); Ari ZIMRAN, Jerusalem (IL)

(73) Assignees: HADASIT MEDICAL RESEARCH SERVICES & DEVELOPMENT LIMITED, Jerusalem (IL); SHAARE ZEDEK SCIENTIFIC LTD., Jerusalem (IL)

(21) Appl. No.: 16/313,683

(22) PCT Filed: Jun. 29, 2017
(86) PCT No.: PCT/IL2017/050730

§ 371 Date: Dec. 27, 2018

Related U.S. Application Data
(60) Provisional application No. 62/356,080, filed on Jun. 29, 2016.

(51) Int. Cl. A61K 31/137; A61K 45/06; A61P 25/28

(52) U.S. Cl. CPC A61K 31/137; A61K 31/7028; A61P 25/28; A61K 45/06

(57) ABSTRACT

Provided are therapeutic combinations of beta-glycolipides and 4-[(2-amino-3,5-dibromophenyl)methylamino]cyclohexan-1-ol, as well as compositions, kits and methods using the same in treating disorders associated with protein misfolding and for immunomodulation.

### FIELD OF THE INVENTION

The invention is in the field of neurology. More specifically, the invention provides novel combinations of beta-glycolipides and 4-[(2-amino-3, 5-dibromophenyl)methylamino]cyclohexan-1-ol, compositions, methods, kits and uses thereof for treating and preventing conditions associated with protein misfolding and protein aggregation, and for immunomodulation.

### BACKGROUND ART

References considered to be relevant as background to the presently disclosed subject matter are listed below:
Babajani G, et al., Pharmacological chaperones facilitate the post-ER transport of recombinant N370S mutant β-glucocerebrosidase in plant cells: evidence that N370S is a folding mutant, Mol. Genet. Metab. 106 (2012) 323-329.
Barker R. A. and Williams-Gray C. H. Review: The spectrum of clinical features seen with alpha synuclein pathology. Neuropathology and Applied Neurobiology (2016), 42, 6-19.
Ben Ya'acov A, et al., Decreased STAT-1 phosphorylation by a thio analogue of beta-D-glucosylceramide is associated with altered NKT lymphocyte polarization, Mol. Immunol. 47 (2009), 526-533.
Breydo, L, et al., A-synuclein misfolding and Parkinson's disease. Biochim. Biophys. Acta 1822 (2012), 261-285.
Gilman S, Wenning G K, Low P A, et al. Second consensus statement on the diagnosis of multiple system atrophy. Neurology (2008), 71: 670-6.
Ilan Y, Compounds of the sphingomyelin-ceramide-glycosphingolipid pathways as secondary messenger molecules: new targets for novel therapies for fatty liver disease and insulin resistance, Am. J. Physiol. Gastrointest. Liver Physiol. 310 (2016) G1102-1117.
Lalazar G. et al., Glycolipids as immune modulatory tools, Mini Rev. Med. Chem. 6 (2006) 1249-1253.
Lalazar G. et al., Modulation of intracellular machinery by beta-glycolipids is associated with alteration of NKT lipid rafts and amelioration of concanavalin-induced hepatitis, Mol. Immunol. 45 (2008) 3517-3525.
Lalazar G, et al., Beta-glycoglycosphingolipid-induced alterations of the STAT signaling pathways are dependent on CDId and the lipid raft protein flotillin-2, Am. J. Pathol. 174 (2009) 1390-1399
Lee M J, et al., Tau degradation: the ubiquitin-proteasome system versus the autophagy lysosome system. Prog Neurobiol 2013; 105: 49-59.
Luan Z, et al., The chaperone activity and toxicity of ambroxol on Gaucher cells and normal mice, Brain Dev. 35 (2013) 317-322.
Margalit M. et al., Glucocerebroside treatment ameliorates ConA hepatitis by inhibition of NKT lymphocytes, Am. J. Physiol. Gastrointest. Liver Physiol. 289 (2005) G917-925.
Margalit M, et al., Glucocerebroside ameliorates the metabolic syndrome in OB/OB mice, J. Pharmacol. Exp. Ther. 319 (2006) 105-110.
McKeith I G, et al., Consortium on DLB. Diagnosis and management of dementia with Lewy bodies: third report of the DLB Consortium. Neurology 65 (2005) 1863-72.
McNeill A, et al. Ambroxol improves lysosomal biochemistry in glucocerebrosidase mutation-linked Parkinson disease cells. Brain 137 (2014) 1481-95.
Narita K, et al., Ambroxol chaperone therapy for neuronopathic Gaucher disease: A pilot study, Ann. Clin. Transl. Neurol. 3 (2016) 200-215.
Sidransky E, et al., The link between the GBA gene and parkinsonism. Lancet Neurol 2012; 11: 986-98.
Suzuki T. et al., Expression of human Gaucher disease gene GBA generates neurodevelopmental defects and ER stress in Drosophila eye, PloS One. 8 (2013).
Williams-Gray C H. et al., The CamPaIGN study of Parkinson's disease: 10-year outlook in an incident population-based cohort. J Neurol Neurosurg Psychiatry 2013 November; 84: 1258-64
Zimran A, et al., Pilot study using ambroxol as a pharmacological chaperone in type 1 Gaucher disease, Blood Cells. Mol. Dis. 50 (2013) 134-137.

Acknowledgement of the above references herein is not to be inferred as meaning that these are in any way relevant to the patentability of the presently disclosed subject matter.

### BACKGROUND OF THE INVENTION

The autophagy-lysosome system plays a key role in degrading misfolded proteins that form the abnormal protein aggregates that occur in the common late onset of neurodegenerative diseases. In concert with the proteasome, the autophagy-lysosome system degrades tau, the proteins that form neurofibrillary tangles in Alzheimer's disease (Lee et al., 2013). There is also strong evidence for a role of this system in the aetiopathogenesis of other neurodegenerative diseases, in particular Parkinson's disease.

It has been recognized that chronic neurodegenerative diseases of the CNS are characterized by the development of intracellular inclusion bodies (Lewy bodies) the main component of which is alpha synuclein protein. Alpha-Synuclein consists of 140 amino acids and is found naturally as an unfolded cytoplasmic protein in neuronal synaptic terminals.

Alpha synuclein is linked to diseases characterized by this type of pathology—namely Parkinson's disease (PD), Dementia with Lewy Bodies (DLB) and multiple system atrophy (MSA) (Breydo. L., 2012). These three disorders are named “alpha synucleinopathies”. Patients with the rare condition Gaucher's disease (GD) are at higher risk of developing alpha synucleinopathy.

Sidransky et al (Sidransky et al., 2012) reported on a relatively high rate of parkinsonism in individuals with mutations in a single allele of the GBA gene. These mutations cause GD when both alleles are mutated due to glucocerebrosidase (GBA) (glucosylceramidase) insufficiency. This enzymatic malfunctioning leads to accumulation of fatty deposits, mainly glucocerebroside (GC), in certain cells and organs. Carriers of GD (only one mutated copy of the gene) are at increased risk of developing PD and other alpha-synucleinopathies even though they do not show any symptoms of GD.

Sidransky et al showed that there seems to be a close inverse relationship between glucocerebrosidase expression-activity and alpha-synuclein accumulation, suggesting that autophagy-lysosome dysfunction is a primary feature of Parkinson's disease pathogenesis, at least for some forms of the disease.

It seems that the increased risk for PD among carriers of GBA mutation is dictated by the type of GBA mutation. For example, carriers of the 84GG null allele have a 15-fold increased risk of PD, whereas carriers of the N370S allele have a 3-fold increased risk of PD.

Surprisingly, however, when it comes to GD patients, most of the cases of PD occur in patients with relatively mild form of GD or in patients who have been taking enzyme replacement therapy (ERT) for many years. PD in patients with severe GD (genotypes N370S/84GG) is very rare. This difference may be related to the level of plasma GC. It appears that while intra-cellular GC is harmful it is beneficial when extracellular, possibly by a protective effect against the inflammatory processes that are associated with various neurodegenerative diseases. If this is the case, a profound decrease in the plasma GC level may result in an added risk of PD and other alpha-synucleinopathies.

The anti-inflammatory effect of GC has been previously demonstrated by the one of the inventors (Ilan, 2016; Lalazar. 2006; Lalazar, 2008; Lalazar, 2009; Margalit, 2005; Margalit, 2006; Ben Ya'acov. 2009). These anti-inflammatory properties were attributed to its potential effects on the cross talk between dendritic-cells (DCs) and natural killer T (NKT) cells.

Ambroxol actions as a chaperone that increases the activity of misfolded GCase was previously demonstrated using in vitro (McNeill. 2014; Babajani, 2012; Luan. 2013) and in vivo (Suzuki. 2013) models. Prevention of neuro-degeneration (McNeill, 2014; Suzuki, 2013) and reversal cellular stress indices were also demonstrated. These studies led to clinical trial in humans. In a pilot study (Narita. 2016), high doses of Ambroxol were given to 5 individuals with type 3 neuropathic GD and debilitating neurological manifestations. Ambroxol led to substantial clinical improvement in both subjective-functional scores and objective scores such as visual evoked potentials (VEP) and ocular movements. Increased GCase activity in lymphocytes and reduction of GSLs in the CSF were also demonstrated. Additional pilot, proof-of-concept study performed by some of the inventors (Zimran, 2013) in patients with the non-neuronopathic form of GD, revealed that ambroxol treatment resulted in disease stability and thus indicated a possible beneficial effect of pharmacologic chaperone therapy.

The long and devastated course of PD and other related disorders require development of the disease modifying therapies that would delay the appearance of the motor symptoms and will prevent cognitive impairments. The present disclosure addresses this need by providing synergistic combinations of compounds accelerating degradation of the misfolded and aggregated proteins. Specifically, this novel treatment strategy targets the occurrence of alpha-synucleinopathies, which underlay the development of neurodegenerative disorders.

### SUMMARY OF THE INVENTION

In a first aspect, the invention relates to a combination comprising at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof, and at least one beta-glycolipid. Specific embodiments relate to combinations of trans-4-(2-Amino-3,5-dibromobenzylamino) cyclohexanol hydrochloride and glucocerebroside.

The invention further provides a composition comprising as an active ingredient a therapeutically effective amount of the combination of the invention.

In another aspect, the invention provides a kit comprising: (i) at least one 4-[(2-amino-3, 5-dibromophenyl)methylamino]cyclohexan-1-ol or any pharmaceutically acceptable salt, solvate, esters, hydrate, stereoisomer or physiologically functional derivative thereof, optionally in a first dosage form: and (ii) at least one beta-glycolipid or any combinations thereof, optionally in a second dosage form.

A further aspect of the invention relates to a method for the treatment, prophylaxis, amelioration, inhibition or delaying the onset of disorders involved in protein misfolding and/or protein aggregation.

The invention further provides the combinations of the invention as well as kits and compositions thereof for use in the method for the treatment, prophylaxis, amelioration, inhibition or delaying the onset of disorders involved in protein misfolding and/or protein aggregation.

These and further aspects of the invention will become apparent by the hand of the following description.

### BRIEF DESCRIPTION OF THE FIGURES

FIG. 1A-1C. oral administration of Ambroxol with β-glucosylceramide (GC) effect on different subsets of lymphocytes

Figure shows the modulation in different subsets of lymphocytes after 5 days of oral administration treatment of Ambroxol in combination with GC compared to monotherapy treatment and control group.

FIG. 1A shows a histogram comparing levels of CD8+CD25+ lymphocytes between mice of different groups.

FIG. 1B shows a histogram comparing CD4/CD8 lymphocyte ratio between mice of different groups.

FIG. 1C shows a histogram comparing intrasplenic/intrahepatic ratio of the CD4/CD8 between mice of different groups.

FIG. 2A-2D. oral administration of Ambroxol with GC shifts expression of pro- to anti-inflammatory cytokines

Figure shows the expression shift of different types of cytokines after 5 days of oral administration treatment of Ambroxol in combination with GC compared to monotherapy treatment and control group.

FIG. 2A shows a histogram comparing expression levels of IL-1α cytokine between mice of different groups.

FIG. 2B shows a histogram comparing expression levels of IL-4 cytokine between mice of different groups.

FIG. 2C shows a histogram comparing expression levels of IFN-γ between mice of different groups.

FIG. 2D shows a histogram comparing expression levels of IL-6 between mice of different groups.

FIG. 3. oral administration of Ambroxol with GC alleviates immune-mediated liver injury

Figure shows a histogram comparing the levels of liver enzymes (ALT and AST) between mice of different groups.

FIG. 4A-4D. oral administration of Ambroxol with GC improves hepatocyte architecture

Figure shows alleviation of liver apoptosis in treated group compared to the untreated group.

FIG. 4A shows representative sections from liver biopsies of the untreated group.

FIG. 4B shows representative sections from liver biopsies of the group which received monotherapy of GC.

FIG. 4C shows representative sections from liver biopsies of the group which received monotherapy of Ambroxol.

FIG. 4D shows representative sections from liver biopsies of the group treated by a combination of GC and Ambroxol.

### DETAILED DESCRIPTION OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In human disorders of impaired protein degradation the result is misfolding and aggregation of intracellular protein with a variety of symptoms that worsen with age. One group of such disorders consists of Parkinson's disease and Parkinson's disease dementia, dementia with Lewy bodies (DLB) and multiple system atrophy (MSA), where abnormal deposits composed mainly of alpha-synuclein are the main etiology.

As discussed herein before, so far, attempts for treatment of PD and related disorders is symptomatic in nature. The main families of drugs useful for treating motor symptoms are levodopa (combined with dopa decarboxylase inhibitor), dopamine agonists, anticholinergic agents and MAO-B inhibitors. Pre-motor (prodromal) symptoms are also treated symptomatically. These pre-motor features include impaired olfaction, constipation, depression and sleepiness. Following the clinical diagnosis of PD and treatment with anti-parkinsonian drugs, side effects such as daytime somnolence, orthostatic hypotension, nausea and gastrointestinal symptoms and hallucinations can appear. In addition, drug-induced involuntary movements (dyskinesia), particularly in the later stages of the disease are common. Dopamine agonists can also induce a number of other behavioral symptoms, such as gambling, hypersexuality, compulsive shopping and compulsive eating and sleep attacks in 14-25% of PD patients. Impaired cognition (mainly executive functions), secondary to the neurodegenerative process is not uncommon in the early stages of the disease and currently is not successfully addressed. These cognitive impairments become more common with the progression of the disease

With disease progression, other neuropsychiatric problems emerge and are often a harbinger of PD. They are typically aggravated by the dopaminergic medication, and by amantadine.

While symptomatic treatment have some efficacy in the earlier stages of the dementia, and can be helpful for the hallucinations and behavioral symptoms as well as cognitive impairment, eventually the disease fails to respond to any treatment. Thus, an effective treatment for controlling the symptoms of PD is missing at the present. Moreover, a treatment to prevent or slow down the development of full blown picture of PD is greatly anticipated.

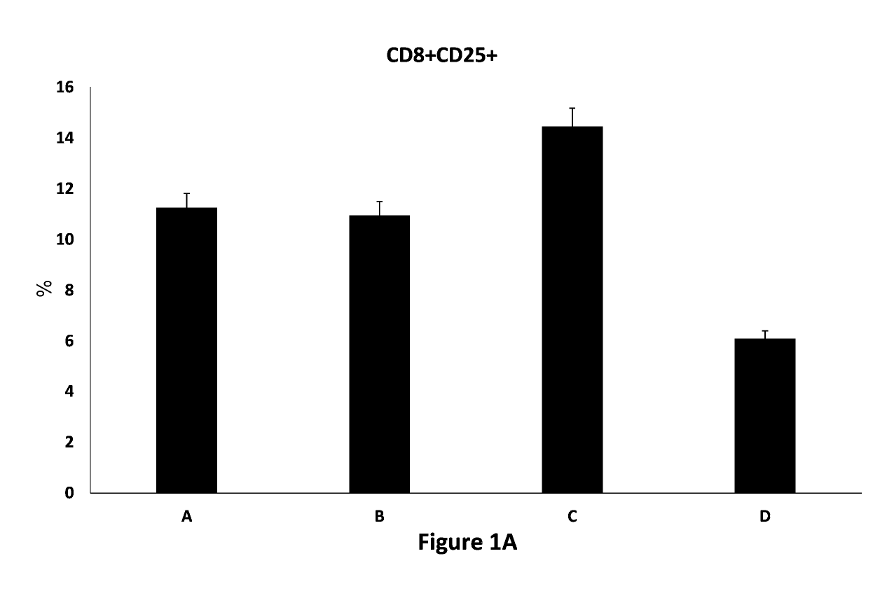

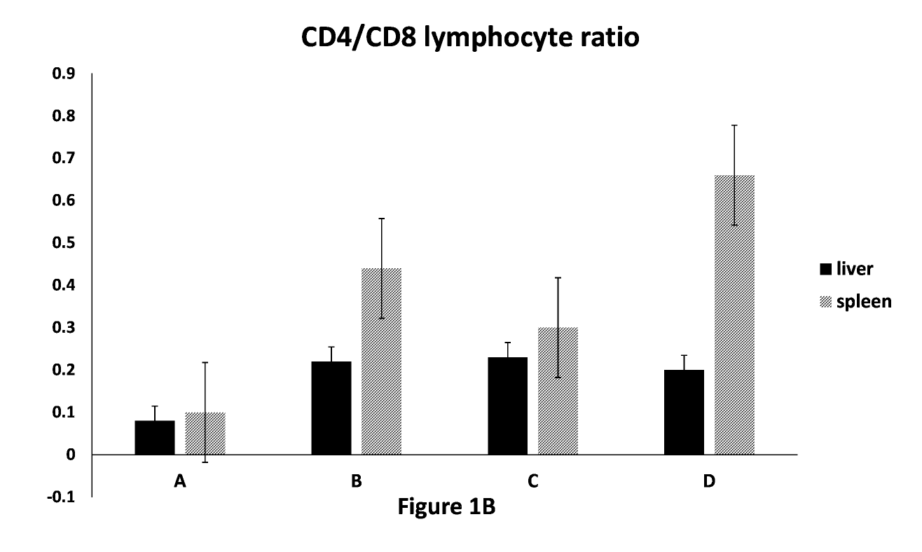

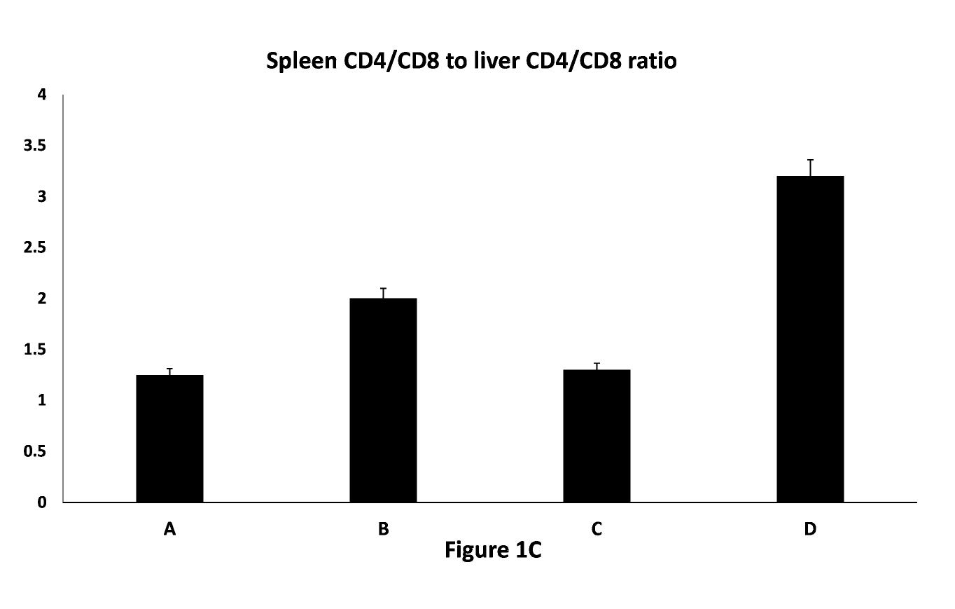

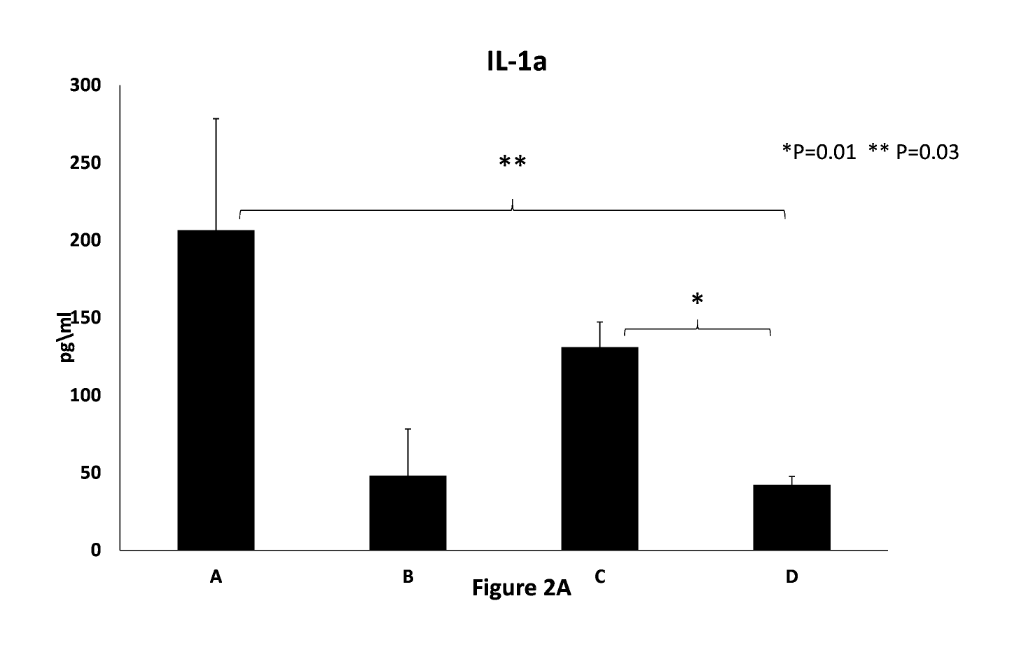

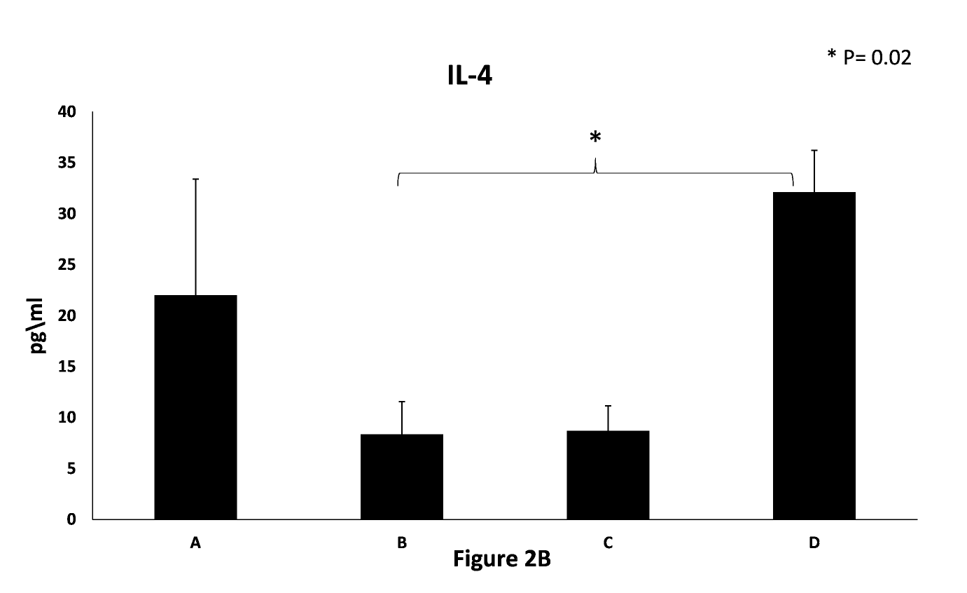

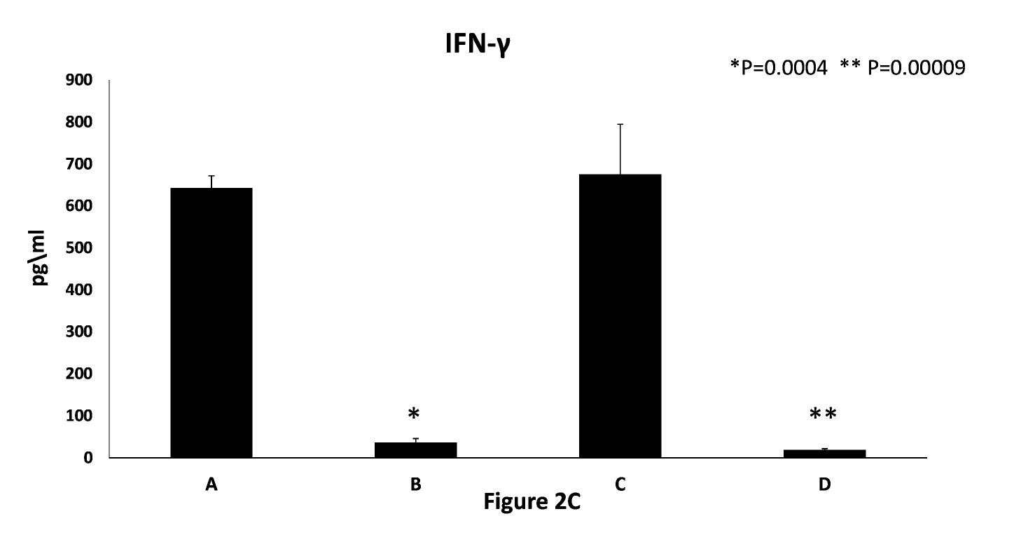

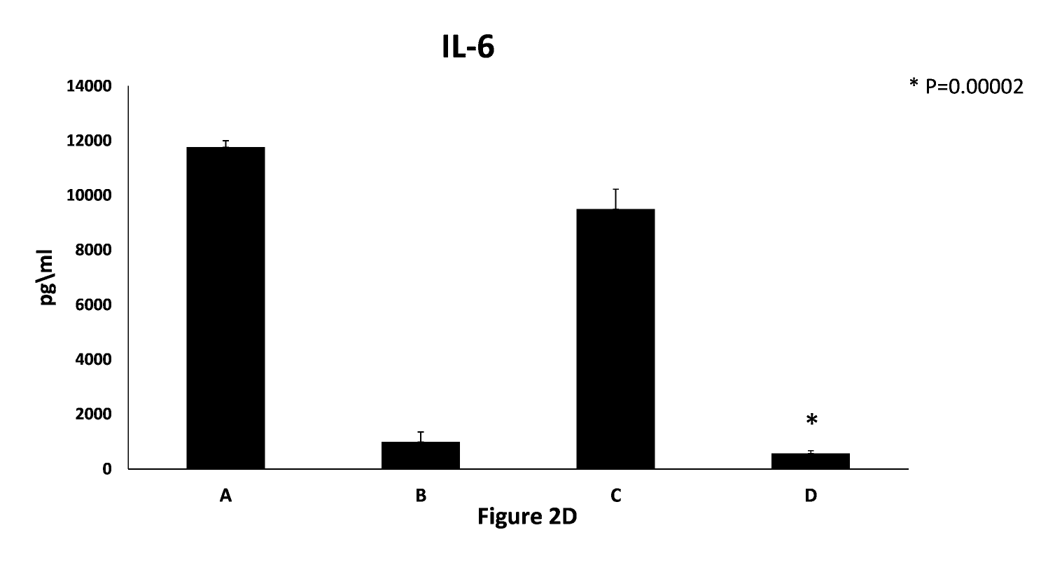

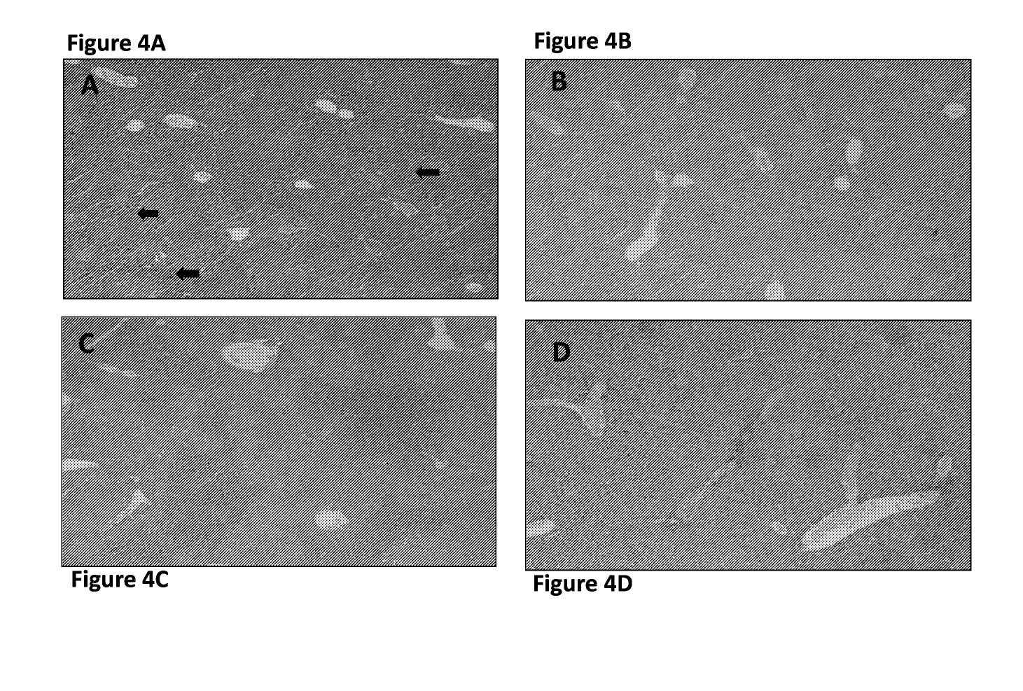

In the present disclosure, the inventors pursue an innovative combined treatment approach to PD, one treatment arm targets cellular defense mechanisms against misfolded and aggregated proteins, pathognomonic for PD and other disorders associated with alpha-symuclein pathology, and the other treatment arm increases the extracellular level of a compound. Moreover, the inventors have demonstrated herein a synergistic anti-inflammatory effect of the novel combination disclosed herein. Modulation of regulatory lymphocytes (FIG. 1), elevation of serum levels of anti-inflammatory cytokines and reduction in the level of pro-inflammatory cytokines (FIG. 2), as well as protection of tissue architecture and thereby reduction in apoptosis (FIG. 4), clearly demonstrate the feasibility of using the immuno-modulatory combination of the invention in the treatment and prevention of neuronal inflammatory conditions.

The results of this approach of the inventors (as shown in Example 1) are highly valuable and may substantiate new therapeutic strategy, which targets both impaired cellular protein degradation in one hand and in the other hand induces extracellular protection.

Thus, according to a first aspect, the invention relates to a combination comprising at least one aminobenzyl-amine aminobenzyl-amine, specifically, 4-[(2-amino-3, 5-dibromophenyl)methylamino]cyclohexan-1-ol, or any pharmaceutically acceptable salt, solvate, esters, hydrate, stereoisomer, physiologically functional derivative thereof, metabolite, enantiomer, stereoisomer, analog, ester, amide or prodrug thereof and at least one beta-glycolipid.

In more specific embodiments, the 4-[(2-amino-3, 5-dibromophenyl)methylamino]cyclohexan-1-ol or any pharmaceutically acceptable salt, solvate, esters, hydrate, stereoisomer or physiologically functional derivative thereof, used in the combination of the invention, may be trans-4-(2-Amino-3,5-dibromobenzylamino) cyclohexanol hydrochloride, specifically, ambroxol.

“Ambroxol” has the IUPAC name of trans-4-(2-Amino-3,5-dibrombenzylamino)-cyclohexanol, as presented by Formula I:

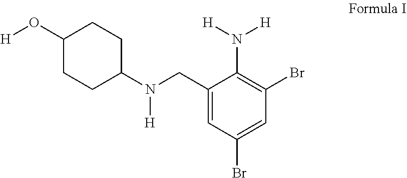 [SMILES: Nc1c(Br)cc(Br)cc1CNC1CCC(O)CC1]

Ambroxol is a clinically used expectorant suggested to act as a ‘chemical’ chaperone facilitating glucosyl ceramidase exit from the endoplasmic reticulum and transport to lysosomes. McNeill et al. Recent findings indicated that treatment with ambroxol hydrochloride increased glucosylceramidase activity and reduced markers of oxidative stress in fibroblasts from healthy controls, Gaucher disease patients with heterozygous glucocerebrosidase mutation with and without Parkinson's disease. It was also shown that ambroxol treatment reduces alpha-synuclein levels in a neuroblastoma line engineered to overexpress alpha-synuclein.

As noted above, the invention also embraces solvates, pharmaceutically acceptable prodrugs, pharmaceutically active metabolites, and pharmaceutically acceptable salts of ambroxol or any variations detailed herein.

The present disclosure also includes any or all of the stereochemical forms, including any enantiomeric or diastereomeric forms, and any tautomers or other forms of ambroxol.

As used herein the term “pharmaceutically acceptable salt” refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, sulfuric acid, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate.

Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a salt of ambroxol.

In some embodiments, the pharmaceutically acceptable salt of ambroxol is a hydrobromide salt. In some further embodiments, the pharmaceutically acceptable salt of ambroxol is hydrochloride salt.

The term “hydrate” refers to a compound formed by the addition of water. The hydrates may be obtained by any known method in the art by dissolving the compounds in water and recrystallizing them to incorporate water into the crystalline structure.

The compounds of the present invention as defined above, may have the ability to crystallize in more than one form, a characteristic, which is known as polymorphism, and it is understood that such polymorphic forms (“polymorphs”) are within the scope the structure described herein. Polymorphism generally can occur as a response to changes in temperature or pressure or both and can also result from variations in the crystallization process. Polymorphs can be distinguished by various physical characteristics known in the art such as x-ray diffraction patterns, solubility, and melting point.

The term “stereoisomer” as used herein is meant to encompass an isomer that possess identical constitution as a corresponding stereoisomer, but which differs in the arrangement of its atoms in space from the corresponding stereoisomer. For example, stereoisomers may be enantiomers, diastereomers and/or cis-trans (E/Z) isomers.

The present disclosure also covers the individual stereoisomer of ambroxol represented, for example, by the following structure of Formula II:

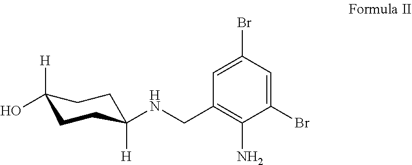

The term “solvate” refers to an aggregate of a molecule with one or more solvent molecules, such as hydrate, alcoholate (aggregate or adduct with alcohol), and the like.

The term “physiologically functional derivative” used herein relates to any physiologically acceptable derivative of ambroxol as described herein.

The physiologically functional derivatives also include prodrugs of ambroxol. Such prodrugs may be metabolized in vivo to a compound of the invention. These prodrugs may or may not be active themselves and are also an object of the present invention.

A “pharmaceutically acceptable prodrug” is a compound that may be converted under physiological conditions to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art and their activities determined using tests such as those described herein. Prodrugs and active metabolites of a compound may be identified using routine techniques known in the art.

As noted above, when referring to ambroxol, it encompass any metabolite or prodrug thereof. Non limiting examples of ambroxol derivative, prodrug or metabolite include bromhexine or any pharmaceutically acceptable salt (2,4-dibromo-6-{[cyclohexyl(methyl)amino]methyl}aniline) having the following structure of Formula III.

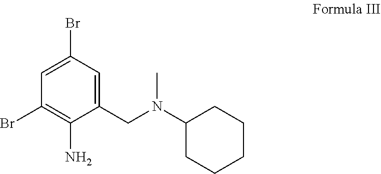 [SMILES: CN(Cc1cc(Br)cc(Br)c1N)C1CCCCC1]

In some embodiments, the salt is a hydrochloride salt.

Yet in some further embodiments, the beta-glycolipid used for the combination of the invention may be at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside, GM3 ganglioside, globoside or any derivative or combinations thereof.

The term “Beta-Glycolipids” or “β-glycolipid ” refers to an abundant and diverse class of lipids in mammalian cells that play important functional roles in membrane structure and signaling. A particularly complex class of glycolipids is the glycosphingolipids, which are composed of a sphingosine backbone linked to a fatty acid and 1 or more hexose sugars. Recent chemical analyses indicate that a typical mammalian cell may contain as many as 10,000 different glycosphingolipids. This structural complexity is generated by an equally elaborate network of biosynthetic and degradative enzymes, many of which remain to be biochemically identified.

The β-glycolipid of the combinations of the invention may be any synthetic or natural β-glycolipid or any derivative or combination thereof. Further, the β-glycolipid of the invention may be selected from the group of glycosphingolipids, of a natural or non-natural source, with any number of cartons and double bonds and with any length of the lipid tail of the molecule. More specifically, the β-glycolipid of the invention may be a glucosylceramide, a monosaccharide ceramide, a galatosylceremide, a lactosyl-ceramide, a gal-gal-glucosyl-ceramide, GM2 ganglioside, GM3 ganglioside, or globoside, or any soy derived product which have been specifically associated with an immunomodulatory effect.

Cerebrosides is the common name for a group of beta-glycosphingolipids called monoglycosylceramides which are important components in animal muscle and nerve cell membranes. They consist of a ceramide with a single sugar residue at the 1-hydroxyl moiety. The sugar residue can be either glucose or galactose; the two major types are therefore called glucocerebrosides and galactocerebrosides. Galactocerebrosides are typically found in neural tissue, while glucocerebrosides are found in other tissues.

The fundamental structure of a cerebroside is ceramide. Monoglycosyl and oligoglycosylceramides having a mono or polysaccharide bonded glycosidically to the terminal OH group of ceramide are defined as cerebrosides. Sphingosine is the main long-chain base present in ceramide. Galactosylceramide is the principal glycosphingolipid in brain tissue. Galactosylceramides are present in all nervous tissues, and can compose up to 2% dry weight of grey matter and 12% of white matter. They are major constituents of oligodendrocytes. Glucosylceramide is found at low levels in animal cells such as the spleen, erythrocytes, and nervous tissues, especially neurons. Glucosylceramide is a major constituent of skin lipids, where it is essential for lamellar body formation in the stratum corneum and to maintain the water permeability barrier of the skin. Glucosylceramide is the only glycosphingolipid common to plants, fungi and animals. It is usually considered to be the principal glycosphingolipid in plants. It is a major component of the outer layer of the plasma membrane. Galactosylceramides have not been found in plants.

Glycosphingolipids are synthesized in the ER and Golgi compartments of the cell and broken down in lysosomes. The functional consequences of this subcellular organization are evident in human genetic diseases in which glycosphingolipid degradation is affected. Loss of any one of a number of catabolic enzymes causes accumulation of glycosphingolipids in the lysosome and a variety of symptoms that worsen with age.

The most common inherited defect in glycosphingolipid breakdown is Gaucher disease (GD), an autosomal-recessive disorder arising from mutations in the gene encoding the lysosomal acid JI-glucosidase acid (GBA or GBA 1).

It was reported that some patients with GD who received enzyme replacement therapy, which resulted in decreased plasma GC level, have developed type 2 diabetes, malignancies and central nervous system disorders.

Since a decrease in plasma GC levels may withhold the protection against the inflammatory processes that are associated with various neurodegenerative diseases, the inventors suggested that an increase in the plasma GC level may decrease a risk for the development of neurogenerative diseases, and as shown herein, specifically for disorders associated with protein misfolding and protein aggregation. Moreover, the inventors report herein that a combined treatment regimen comprising ambroxol and GC results in surprising immunomodulatory synergistic effect and may thus be applicable in patients suffering from neurodegenerative disorders associated with protein misfolding and protein aggregation, specifically, disorders associated with characterized by alpha synuclein pathology.

As noted above, it should be appreciated that the invention provides the use of synthetic as well as naturally occurring beta glycolipides.

The combination of the invention takes advantage of the fact that on one hand ambroxol is able to reduce impaired intracellular protein aggregation, in particular alpha-synuclein, and on the other hand glucocerebroside, that is the glycosphingolipid that accumulates in this disease may exert a neuroprotective effect as it is known that patients with Guacher disease who receive enzyme therapy have higher prevalence of Parkinson. However, the synergistic effect of this combination on inflammation and thus its applicability for cognitive decline and neuroprotection is surprising. Therefore, in some specific embodiments, the invention provides a combination comprising trans-4-(2-Amino-3,5-dibromobenzylamino) cyclohexanol hydrochloride (ambroxol) and glucocerebroside (GC).

In yet some further embodiment, the combination of the invention may further comprise at least one additional therapeutic drug.

A further aspect the invention provides a composition comprising an effective amount of a combination of at least one 4-[(2-amino-3, 5-dibromophenyl)methylamino]cyclohexan-1-ol or any pharmaceutically acceptable salt, solvate, hydrate, stereoisomer or physiologically functional derivative thereof, and at least one beta-glycolipid or any derivatives or analogs thereof. The composition of the invention may optionally further comprise at least one of pharmaceutically acceptable carrier/s, diluents/s, excipient/s, diluent/s, additive/s and adjuvant/s.

In some particular embodiments, the at least one 4-[(2-amino-3, 5-dibromophenyl)methylamino]cyclohexan-1-ol or any pharmaceutically acceptable salt, solvate, hydrate, stereoisomer or physiologically functional derivative thereof, used for the composition of the invention, may be trans-4-(2-Amino-3,5-dibromobenzylamino) cyclohexanol hydrochloride, specifically, ambroxol.

In yet some other embodiments, the beta-glycolipid may be at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside. GM3 ganglioside, globoside or any derivative or combinations thereof.

In some specific embodiments, the composition of the invention may comprise an effective amount of trans-4-(2-Amino-3,5-dibromobenzylamino) cyclohexanol hydrochloride, specifically ambroxol and glucocerebroside.

It should be appreciated that the at least one ambroxol and at least one glucocerebroside may be presented in the compositions, as well as in the combinations and kits of the invention at any ratio, for example, 1:1, to 0.0001-100,000 or more. More specifically, 0.1:1, 0.2:1, 0.3:1, 0.4:1, 0.5:1, 0.6:1, 0.7:1, 0.8:1, 0.9:1, 1:1, 1:2, 1:3, 1:4, 1:5, 1:6, 1:7, 1:8, 1:9, 1:10, or more. As a non-limiting example, a quantitative ratio used between any of the compounds may be: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1500, 1:750, 1:1000. It should be further noted that where the combination of the invention comprises more than two compounds, the quantitative ratio used may be for example, 1:1:1, 1:2:3, 1:10:100, 1:10:100:1000 etc.

It should be appreciated that the composition of the invention may comprise any derivative or formulation of said ambroxol and glucocerebroside, specifically, any of the derivatives disclosed herein.

In some specific embodiments, the combination of the invention may further comprise at least one additional therapeutic drug. In more specific embodiments, such drug may be any drug displaying a neuroprotective effect. In yet more specific embodiments, such drug may be any drug or any combination of drugs used for the treatment of neurodegenerative disorder/s. To name but few, such drugs may include but are not limited to drugs used for treating PD. In more specific embodiments, such drugs may include Levodopa, levodopa combined with carbidopa (Rytary, Sinemet), specifically, Carbidopa-levodopa, Dopamine agonists such as pramipexole (Mirapex), ropinirole (Requip) and rotigotine (given as a patch, Neupro), a short-acting injectable dopamine agonist, apomorphine (Apokyn), monoamine oxidase B (MAO-B) inhibitors that include selegiline (Eldepryl. Zelapar) and rasagiline (Azilect), Catechol-O-methyltransferase (COMT) inhibitors, such as Entacapone (Comtan) and Tolcapone (Tasmar), Anticholinergics that include benztropine (Cogentin) or trihexyphenidyl, and Amantadine. It should be further understood that any additional drug that exhibits any neuroprotective effect, for example, drugs used for treating any neurodegenerative or any protein misfolding disorder, such as Parkinson, or Alzheimer disease, is encompassed by the invention.

In some embodiments, the compositions of the invention may be formulated in a pharmaceutical composition. More specifically, the composition of the invention may comprise a therapeutically effective amount of at least one of the trans-4-(2-Amino-3,5-dibromobenzylamino) cyclohexanol hydrochloride, specifically, ambroxol and a therapeutically effective amount of the at least one of the beta-glycolipides, specifically, glucocebroside as described above, and at least one of pharmaceutically acceptable carrier/s, diluent/s, excipient/s. In some particular embodiments, the invention provides a pharmaceutical composition comprising trans-4-(2-Amino-3,5-dibromobenzylamino) cyclohexanol hydrochloride and glucocebroside at any ratio.

In further specific and non-limiting embodiments, the pharmaceutical composition of the invention may be applicable in the treatment, prophylaxis, amelioration, inhibition or delaying the onset of disorders involved in protein misfolding and protein aggregation, or of any early signs or symptoms associated therewith.

“Protein misfolding and aggregation” as used herein, relates to an impaired physical process by which a protein chain acquires its native three-dimensional structure, a conformation that is usually biologically functional, in an expeditious and reproducible manner. It is the physical process by which a polypeptide folds into its characteristic and functional three-dimensional structure from random coil. Each protein exists as an unfolded polypeptide or random coil when translated from a sequence of mRNA to a linear chain of amino acids. Amino acids interact with each other to produce a well-defined three-dimensional structure, the folded protein, known as the native state. The correct three-dimensional structure is essential to function, although some parts of functional proteins may remain unfolded. Failure to fold into native structure generally produces inactive proteins, but in some instances misfolded proteins have modified or toxic functionality. Several neurodegenerative and other diseases are believed to result from the accumulation of amyloid fibrils formed by misfolded proteins.

More specifically, under some conditions, proteins may not fold into their biochemically functional forms resulting in protein denaturation. A fully denatured protein lacks both tertiary and secondary structure, and exists as a so-called random coil. Under certain conditions some proteins can refold; however, in many cases, denaturation is irreversible. Cells sometimes protect their proteins against the denaturing influence of heat with enzymes known as chaperones or heat shock proteins, which assist other proteins both in folding and in remaining folded. Some proteins never fold in cells at all except with the assistance of chaperone molecules, which either isolate individual proteins so that their folding is not interrupted by interactions with other proteins or help to unfold misfolded proteins, giving them a second chance to refold properly. This function is crucial to prevent the risk of precipitation into insoluble amorphous aggregates.

Aggregated proteins are associated with prion-related illnesses such as Creutzfeldt-Jakob disease, bovine spongiform encephalopathy (mad cow disease), amyloid-related illnesses such as Alzheimer's disease and familial amyloid cardiomyopathy or polyneuropathy, as well as intracytoplasmic aggregation diseases such as Huntington's and Parkinson's disease. These age onset degenerative diseases are associated with the aggregation of misfolded proteins into insoluble, extracellular aggregates and/or intracellular inclusions including cross-beta sheet amyloid fibrils. It is not completely clear whether the aggregates are the cause or merely a reflection of the loss of protein homeostasis, the balance between synthesis, folding, aggregation and protein turnover. Misfolding and excessive degradation instead of folding and function leads to a number of proteopathy diseases such as antitrypsin-associated emphysema, cystic fibrosis and the lysosomal storage diseases, where loss of function is the origin of the disorder.

As some of the conditions associated with protein missfolding and protein aggregations involve neurodegeneration, in certain specific embodiments, the pharmaceutical composition of the invention may be applicable in the treatment of neurodegenerative diseases.

The term “neurodegenerative diseases” is the general term for the progressive loss of structure or function of neurons, leading to their death. The greatest risk factor for neurodxiegenerative diseases is aging. Mitochondrial DNA mutations as well as oxidative stress both contribute to aging. Many of these diseases are late-onset, meaning there is some factor that change as a person ages, for each disease. One constant factor is that in each disease, neurons gradually lose function as the disease progresses with age.

Thus, the present disclosure provides the pharmaceutical composition for use in the treatment of neurodegenerative disorders, in particular disorders of alpha-synuclein pathology.

“Alpha-syniclein pathology disorders” as used herein are disorders characterized by the presence of a specific intracellular protein aggregates (inclusion bodxiies) known as Lewy bodies that contain mainly alpha-synuclein protein. Alpha-synuclein protein consists of 140 amino acids and is found naturally as an unfolded cytoplasmic protein in neuronal synaptic areas.

Overexpression of alpha-synuclein interrupts normal cell functions and leads to decreases in neurite outgrowth and cell adhesion. Alpha-synuclein aggregates comprising monomeric, oligomeric intermediate, or fibrillar forms are thought to be involved in a critical step in the pathogenesis of Parkinson's disease (PD) and in other alpha-synucleinopathies, such as multiple system atrophy (MSA) and dementia with Lewy bodies (DLB). These chronic neurodegenerative diseases of the CNS are characterized by the development of Lewy bodies containing alpha-synuclein protein. Oligomeric and monomeric alpha-synuclein have both been detected in cerebrospinal fluid and plasma samples from PD patients, suggesting that small aggregates of alpha-synuclein access the extracellular space. Previous animal and clinical data suggest that misfolded alpha-synuclein can be released from cells by exocytosis and transmitted from one brain area to another via cell-to cell propagation. Although the exact mechanism of alpha-synuclein transmission remains unknown, evidence suggests that clathrin-mediated endocytosis (CME) may have an important role in internalization of extracellular a-synuclein. As the cargo protein for endocytosis is usually recognized by a specific receptor on the cell surface, it is possible that alpha-synuclein may interact with cell-surface receptors that have not been well specified until now. N-methyl-D-aspartate (NMDA) receptor subunits contain motifs that bind the endocytic adaptor protein involved in CME. Additionally, a recent study provided the evidence that alpha-synuclein could promote endocytic internalization of surface NMDA receptors through a mechanism requiring clathrin, suggesting an interaction between alpha-synuclein and NMDA receptors. Accordingly, alpha-synuclein propagation from one area of the brain to others via cell-to-cell transmission is closely related with disease progression or clinical severity. Thus, strategies targeting modulation of alpha-synuclein transmission may be important for the development of future disease-modifying therapies in individuals with alpha-synucleinopathies.

In some specific embodiments, the pharmaceutical composition of the invention may be applicable for use in the treatment of at least one of Parkinson's disease (PD). Dementia with Lewy Bodies (DLB) and multiple system atrophy (MSA).

In yet some further embodiments, the pharmaceutical composition may be intended for use in the treatment of Parkinson disease and/or any symptoms or conditions, any dementia or cognitive decline associated therewith.

In some specific embodiments, the pharmaceutical composition of the invention may be particularly applicable in the treatment of DLB.

In further specific embodiments, the pharmaceutical composition of the invention may be applicable in the treatment of MSA.

Still further, it should be appreciated that in certain embodiments, the pharmaceutical composition of the invention may be further applicable for treating disorders characterized by beta-amyloid protein aggregation.

A group of disorders associated with beta-amyloid protein aggregation include Alzheimer's disease (AD), where deposits of a protein precursor called beta-amyloid build up (termed plaques) in the spaces between nerve cells and twisted fibers of tau protein build up (termed tangles) inside the cells.

More specifically, “Beta-amyloid protein aggregations” as used herein relates to cerebral plaques laden with 3-amyloid peptide (All) and dystrophic neurites in neocortical terminal fields as well as prominent neurofibrillary tangles in medial temporal-lobe structures, which are important pathological features of Alzheimer's disease. Subsequently, loss of neurons and white matter, congophilic (amyloid) angiopathy are also present.

Aβ peptides are natural products of metabolism consisting of 36 to 43 amino acids. Monomers of Aβ40 are much more prevalent than the aggregation-prone and damaging Aβ42 species. β-amyloid peptides originate from proteolysis of the amyloid precursor protein by the sequential enzymatic actions of beta-site amyloid precursor protein-cleaving enzyme 1 (BACE-1), a β-secretase, and γ-secretase, a protein complex with presenilin 1 at its catalytic core. An imbalance between production and clearance, and aggregation of peptides, causes Aβ to accumulate, and this excess may be the initiating factor in Alzheimer's disease.

β-amyloid can also grow into fibrils, which arrange themselves into β-pleated sheets to form the insoluble fibers of advanced amyloid plaques. Soluble oligomers and intermediate amyloid are the most neurotoxic forms of Aβ. In brain-slice preparations, dimers and trimers of Aβ are toxic to synapses. Experimental evidence indicates that Aβ accumulation precedes and drives tau protein aggregation.

“Tau protein” as used herein, refers to neurofibrillary tangles, which are filamentous inclusions in pyramidal neurons, characteristic for Alzheimer's disease and other neurodegenerative disorders termed tauopathies. Elucidation of the mechanisms of their formation may provide targets for future therapies. Accumulation of hyperphosphorylated Tau protein as paired helical filaments in pyramidal neurons is a major hallmark of Alzheimer disease (AD). Besides hyperphosphorylation, other modifications of the Tau protein, such as cross-linking, are likely to contribute to the characteristic features of paired helical filaments, including their insolubility and resistance against proteolytic degradation. These neurofibrillary tangles, consist of hyperphosphorylated and aggregated forms of the microtubule-associated protein tau.

Under nonpathological conditions, tau is a developmentally regulated phosphoprotein that promotes assembly and stability of microtubules and is thus involved in axonal transport. In AD and other tauopathies, tau proteins aggregate and form fibrillar insoluble intracellular inclusions, so-called neurofibrillary tangles. It has been suggested that ionic interactions and covalent cross-linking contribute to pathological Tau aggregation and tangle formation. Reactive carbonyl compounds, which are increased under conditions of oxidative stress and in aging have been proposed as potential compounds responsible for tau aggregation.

Still further, as noted herein above, the invention provides combinations, compositions, kits and methods applicable in protecting against any neurodegeneration. Neurodegeneration is a common theme of many nervous system diseases and disorders, such as Parkinson's disease, Alzheimer's disease, ALS, head trauma, epilepsy and stroke. These disorders are devastating and their management expensive, with annual costs currently exceeding several hundred billion dollars in the United States alone, and current treatments are inadequate. Adding to the urgency of the problem is the fact that the incidence of these age-related disorders is increasing rapidly as population demographics change.

A common theme of these diseases and disorders is the loss of neural cell functions and/or neural cell death or damage. Here, the inventors disclose composition and methods involving exposing neural cells, whether directly or through administration to a patient, to a combination of at least one beta glycolypide and ambroxol, for immune-modulation and neuro-protection and thereby prevention and treatment of pathologies which cause neural cell deterioration and death.

When referring to cell damage, the term “damage” relates to any disruption of physiological cell functions or cell death. Non-limiting examples for disruption of physiological cell functions include: oxidative stress (for example, lipid peroxidation, DNA and RNA oxidation and protein oxidation), non-specific glycation, protein misfolding, DNA mutation, loss of any cellular structure integrity, metabolic stress, ionizing and non-ionizing radiation damage and chemical stress (for example, exposure to acid or basic substances).

Accordingly, the expression “protection from neural cell damage or deterioration in neural cell function” means either preventing or decreasing neural death, or preventing or decreasing the deterioration in neural function (as exemplified for instance by secretion of neurotransmitters, dendrite and axonal growth, transfer of electrical impulses, response to stimuli, maintaining structural integrity of myelin sheaths and Ranvier's nodes, etc.)

The expression “reduction” and “inhibition” of neural cell damage or deterioration in neural cell function, or decrease in neural cell death or neural function loss, relate to the retardation, lessening or attenuation of a process which inflicts neural cell damage and/or affects neural cell activity detrimentally. Such reduction includes reduction by any one of about 1% to about 99%, 2% to about 98%, 3% to about 97%, 4% to about 96%, 5% to about 95%, 6% to about 94%, 7% to about 93%, 8% to about 92%, 9% to about 91%, 10% to about 90% or 11% to about 89%, 12% to about 88%. 13% to about 87%. 14% to about 86%, 15% to about 85%, 16% to about 84%, 17% to about 83%. 18% to about 82%, 19% to about 81%, 20% to about 80%. 21% to about 79%, 22% to about 78%, 23% to about 77%, 24% to about 76%, 25% to about 75%, 26% to about 74%. 27% to about 73%, 28% to about 72%. 29% to about 71%, 2% to about 70%. 32% to about 69%. 33% to about 68%, 34% to about 67%, 35% to about 66%, 36% to about 65%, 37% to about 64%, 38% to about 63%, 39% to about 62%, 40% to about 61%, 41% to about 60%, 42% to about 59%, 43% to about 58% or 44% to about 57%. More specifically, such reduction includes reduction by any one of about 1%, 5%, 10%, 15%, 20%, 25%, 30%, 35%, 40%, 45%, 50%. 55%, 60%, 65%. 70%, 75%, 80%. 85%, 90%, 95% or 100%.

Moreover, with regards to the above, it is to be understood that, where provided, percentage values such as, for example, 10%. 50%, 120%, 500%, 1000%, 2000% etc., are interchangeable with “fold change” values, i.e., 0.1, 0.5, 1.2, 5, etc., respectively.

The term “neural cell function” relates to any normal physiological cellular activity, depending on the specific cell type. Non-limiting examples of such functions include cell viability, secretion of neurotransmitters, dendrite and axonal growth, transfer of electrical impulses and response to stimuli in neurons, maintaining structural integrity of myelin sheaths and Ranvier's nodes in oligodendrocytes and Schwann cells, and supplying nutrients and oxygen, and recycling neurotransmitters in astrocytes.

It should be appreciated that throughout this specification, the term “neural cell” relates to cells that may be any one of central nervous system neurons and glial cells, astrocyte, neuron cells, oligodendrocyte. Schwann cells, satellite cells, spindle cells, neuronauditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells of eye, photoreceptor green-sensitive cone cells of eye, photoreceptor red-sensitive cone cells of eye, proprioceptive primary sensory neurons, touch-sensitive primary sensory neurons, type I carotid body cells, type II carotid body cells, type I hair cells of vestibular apparatus of ear, type II hair cells of vestibular apparatus of ear, type I taste bud cells, autonomic neuron cells, cholinergic neural cells, adrenergic neural cells, peptidergic neural cells, sense organ and peripheral neuron supporting cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells and enteric glial cells.

Since the invention provides methods for protection from, reduction, prevention or inhibition of deterioration in neural cell function in a subject in need thereof, it is important to clearly define the scope of the term “neural cell function”. Herein, this term relates to any normal physiological cellular activity, depending on the specific cell type. Non-limiting examples of such functions include cell viability, secretion of neurotransmitters, dendrite and axonal growth, transfer of electrical impulses and response to stimuli in neurons, maintaining structural integrity of myelin sheaths and Ranvier's nodes in oligodendrocytes and Schwann cells, and supplying nutrients and oxygen, and recycling neurotransmitters in astrocytes.

It should be therefore appreciated that the pharmaceutical compositions of the invention as well as all combinations described above may be applicable for any of the neurodegenerative disorders discussed above, specifically, to any of the alpha-synuclein pathologies, any conditions associated with aggregation of beta-amyloid, any of the taupathies mentioned above and/or any early signs or symptoms associated therewith.

As noted herein above, the invention provides pharmaceutical compositions suitable for treating the disorders disclosed above. “Pharmaceutical compositions” according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, and suitable mixtures thereof. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings and the like. The use of such media and agents for pharmaceutical active substances is well known in the art. Except as any conventional media or agent is incompatible with the active ingredient, its use in the therapeutic composition is contemplated.

In yet further embodiments the pharmaceutical composition of the invention may be suitable for systemic administration. The pharmaceutical composition of the invention can be administered and dosed by the methods of the invention, in accordance with good medical practice. More specifically, the compositions used in the methods and kits of the invention, described herein after, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

According to certain embodiments, the combination of the invention or any composition or kit thereof may be administered by oral, intravenous, intramuscular, subcutaneous, intraperitoneal, perenteral, transdermal, intravaginal, intranasal, mucosal, sublingual, topical, rectal or subcutaneous administration, or any combination thereof.

According to a specific embodiment, the composition of the invention may be particularly suitable for oral or mucosal administration. The usefulness of an oral formulation requires that the active agent or combinations of the invention be bio-available. Bioavailability of orally administered drugs can be affected by a number of factors, such as drug absorption throughout the gastrointestinal tract, stability of the drug in the gastrointestinal tract, and the first pass effect.

Thus, effective oral delivery of an active agent or combination requires that the active agent have sufficient stability in the stomach and intestinal lumen to pass through the intestinal wall. Many drugs, however, tend to degrade quickly in the intestinal tract or have poor absorption in the intestinal tract so that oral administration is not an effective method for administering the drug.

More specifically, the composition of the invention may be suitable for mucosal administration, for example, pulmonary, buccal, nasal, intranasal, sublingual, rectal, vaginal administration and any combination thereof.

Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home.

Liquid dosage forms also allow subjects to easily take the required dose of active ingredient. Liquid preparations can be prepared as a drink, or to be administered, for example, by a nasal-gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions of the invention can also optionally be formulated to reduce or avoid the degradation, decomposition, or deactivation of the active agents by the gastrointestinal system, e.g., by gastric fluid in the stomach. For example, the compositions can optionally be formulated to pass through the stomach unaltered and to dissolve in the intestines, i.e., enteric coated compositions.

Oral compositions can also be prepared using an excipient. Pharmaceutically compatible binding agents, and/or adjuvant materials can be included as part of the composition. Oral dosage forms comprising combined beta-glycolipid and ambroxol are provided, wherein the dosage forms, upon oral administration, provide a therapeutically effective blood level of the combined beta-glycolipid and ambroxol to a subject. Also provided are dosage forms comprising said combination wherein the dosage forms, upon administration, provide a therapeutically effective blood level of the combined beta-glycolipid and ambroxol to a subject. For the purpose of mucosal therapeutic administration, the active combined compounds (e.g., beta-glucosylceramide with ambroxol) can be incorporated with excipients or carriers suitable for administration by inhalation or absorption, e.g., via nasal sprays or drops, or rectal or vaginal suppositories.

Solid oral dosage forms include, but are not limited to, tablets (e.g., chewable tablets), capsules, caplets, powders, pellets, and granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e.g., with a suitable polymer or a conventional coating material to achieve, for example, greater stability in the gastrointestinal tract, or to achieve a desired rate of release.

In addition, a capsule comprising the powder, pellets or granules can be further coated. A tablet or caplet can be scored to facilitate division for ease in adjusting dosage as needed.

The dosage forms of the present invention can be unit dosage forms wherein the dosage form is intended to deliver one therapeutic dose per administration, e.g., one tablet is equal to one dose. Such dosage forms can be prepared by methods of pharmacy well known to those skilled in the art. Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents. Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidinones, methyl cellulose, pro-gelatinized starch, hydroxypropyl methyl cellulose, microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions.

Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolate, corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or Sterotes, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof.

The pharmaceutical compositions and oral or mucosal dosage forms can further comprise one or more compounds that reduce the rate by which an active ingredient will decompose. Thus the oral dosage forms described herein can be processed into an immediate release or a sustained release dosage form. Immediate release dosage forms may release the combined beta-glycolipid and ambroxol in a fairly short time, for example, within a few minutes to within a few hours. Sustained release dosage forms may release the combined beta-glycolipid and ambroxol over a period of several hours, for example, up to 24 hours or longer, if desired. In either case, the delivery can be controlled to be substantially at a certain predetermined rate over the period of delivery. In some embodiments, the solid oral dosage forms can be coated with a polymeric or other known coating material(s) to achieve, for example, greater stability on the shelf or in the gastrointestinal tract, or to achieve control over drug release. Such coating techniques and materials used therein are well-known in the art. Such compounds, which are referred to herein as “stabilizers,” include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and so hydroxypropylmethyl cellulose acetate succinate, among others, can be used to achieve enteric coating. Mixtures of waxes, shellac, rein, ethyl cellulose, acrylic resins, cellulose acetate, silicone elastomers can be used to achieve sustained release coating.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, or elixir of the active ingredient in the liquid. The solutions, suspensions, syrups, and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring. Sweeteners can include sugars, aspartame, saccharin, sodium cyclamate and xylitol.

For administration by inhalation, the combined beta-glycolipid and ambroxol compounds can be delivered in the form of an aerosol spray from pressured container or dispenser which contains a suitable propellant, e.g., a gas such as carbon dioxide, or a nebulizer.

Dosage, toxicity and therapeutic efficacy of such combined beta-glycolipid and ambroxol compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between so toxic and therapeutic effects is the therapeutic index and it can be expressed as the ratio LD50/ED50. Compositions which exhibit high therapeutic indices are preferred.

The combined compounds of the present invention are generally administered in the form of a pharmaceutical composition comprising both compounds of this invention together with a pharmaceutically acceptable carrier or diluent. Alternatively, both compounds may be administered in separate compositions. Thus, the compounds used by this invention can be administered either individually in a kit or together in any conventional dosage form.

More particularly, the present invention relates to combined therapy involving the use of at least two compounds, specifically, trans-4-(2-Amino-3,5-dibromobenzylamino) cyclohexanol hydrochloride (ambroxol) and glucocebroside that may be administered either together in a pharmaceutical composition, or in separate compositions through different routes, dosages and combinations. More specifically, the treatment of diseases and conditions with a combination of active ingredients may involve separate administration of each active ingredient. Therefore, a kit providing a convenient modular format of the different constituents of the compounds and related components required for treatment would allow the required flexibility in the above parameters.

Thus, in another aspect, the invention provides a kit.

The kit of the invention may comprise: (i) at least one 4-[(2-amino-3, 5-dibromophenyl)methylamino]cyclohexan-1-ol or any pharmaceutically acceptable salt, solvate, esters, hydrate, stereoisomer or physiologically functional derivative thereof, optionally in a first dosage form: and (ii) at least one beta-glycolipid or any combinations thereof, optionally in a second dosage form.

In some specific embodiments, the kit of the invention may comprise trans-4-(2-Amino-3,5-dibromobenzylamino) cyclohexanol hydrochloride, specifically, ambroxol.

In yet some further embodiments, the kits of the invention may comprise at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside, GM3 ganglioside, globoside or any derivative or combinations thereof.

Still further, in some embodiments, the kits of the invention may comprise trans-4-(2-Amino-3,5-dibromobenzylamino) cyclohexanol hydrochloride, specifically ambroxol, and glucocerebroside.

In some specific embodiments, the kit of the invention may further comprise at least one additional therapeutic drug, specifically a drug displaying a neuroprotective effect. In more specific embodiments, the additional drug may be any of the drugs used for treating PD or Alzheimers disease disclosed herein before.

More specifically, the kit may further include container means for containing both separate compositions, such as a divided bottle or a divided foil packet. However, the separate compositions may also be contained within a single, undivided container. Typically the kit includes directions for the administration of the separate components. As noted above, the kit form is particularly advantageous when the separate components are preferably administered in different dosage forms (e.g., parenteral vs. topical), are administered at different dosage intervals, or when titration of the individual components of the combination is desired by the prescribing physician.

According to some embodiments, the kit of the invention are intended for achieving a therapeutic effect in a subject suffering from disorders associated with protein misfolding and protein aggregations as described above. It should be further appreciated that the kit of the invention may be also used for preventing the described disorders in subjects having an increased risk and/or subjects displaying early signs or symptoms.

Achieving a therapeutic effect is meant for example, where the kit is intended for the treatment of a specific disorder, such as PD, DLB and MSA or any conditions associated therewith, the therapeutic effect may be for example slowing the progression of the treated condition.

The invention provides a method of treating, ameliorating, preventing or delaying the onset of disorders associated with protein misfolding and protein aggregations in a subject in need thereof using the unit dosage forms comprised in a kit according to the invention. Thus, in some embodiments, the kit of the invention enables the use of the active ingredients in a method of treating, preventing, inhibiting, reducing, eliminating, protecting or delaying the onset of a pathological conditions or a disorders associated with protein misfolding and protein aggregation or any early signs or symptoms associated therewith.

In certain specific embodiments, the kit of the invention may be used in the treatment of neurodegenerative disorders. In certain specific embodiments, the kit of the invention may be used in the prevention of neurodegenerative disorders in patients who are at risk for the disease In yet some other particular and non-limiting embodiments, the invention further provides the kit for use in the treatment of disorders characterized by alpha-synuclein pathology.

In some embodiments, the kit of the invention may be suitable for use in the treatment, prophylaxis, amelioration, inhibition or delaying the onset of at least one of Parkinson disease, Dementia with Lewy Bodies (DLB) and multiple system atrophy (MSA) and/or any early signs or symptoms associated therewith.

In one embodiment the kit of the invention may be used in the treatment, prophylaxis, amelioration, inhibition or delaying the onset of Parkinson disease and any cognitive decline, dementia, and/or any early signs or symptoms associated therewith.

In yet some other embodiments, the kit of the invention may be used in the treatment, prophylaxis, amelioration, inhibition or delaying the onset of DLB and/or any early signs or symptoms associated therewith.

Still further, in some embodiments the kit of the invention may be used in the treatment, prophylaxis, amelioration, inhibition or delaying the onset of MSA and/or any early signs or symptoms associated therewith.

In some specific embodiments, the kit of the invention may be used in the treatment of disorders characterized by beta-amyloid protein aggregation.

In further embodiments the kit is for use in a treatment of at least one of Alzheimer's and age-associated cognitive decline (ACD) or mild cognitive impairment (MCI).

It should be understood that the kit of the invention may be used in some embodiments of the invention for treating subjects suffering from a pathological disorders or diseases as outlined above. It should be further noted that the application of the kit of the invention or any component thereof, may form a complementary treatment regimen for subjects suffering from any of the pathological disorders or diseases as discussed above.

A further aspect of the invention relates to a method for the treatment, prophylaxis, amelioration, inhibition or delaying the onset of disorders involved in protein misfolding and/or protein aggregation. More specifically, the method of the invention may comprise the step of administering to a subject in need thereof an effective amount of at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof and at least one beta-glycolipid, and derivatives thereof or any combinations thereof, or any compositions or kits comprising the same.

It should be appreciated that the combinations of the invention, compositions, kits and methods described by the invention, may be applicable for any form of protein misfolding disorder, specifically, any form of protein aggregation disclosed herein.

In some specific embodiments, the methods of the invention employ trans-4-(2-Amino-3,5-dibromobenzylamino) cyclohexanol hydrochloride, specifically, ambroxol.

In other embodiments, the methods of the invention employ as a natural or synthetic beta-glycolipid, at least one of glucocerebroside, glucosylceramide, glucosylsphingosine, lactosylceramide, glycosphingolipid, monosaccharide ceramide, galatosylceremide, gal-gal-glucosyl-ceramide. GM2 ganglioside. GM3 ganglioside, globoside or any derivative or combinations thereof.

In more specific embodiments, the method of the invention may comprise the step of administering to the treated subject a therapeutically effective amount of ambroxol and glucocerebroside, or any combinations thereof, or any compositions or kits comprising the same.

Still further in some embodiments the combined therapy disclosed by the present invention further pertains to therapeutic methods involving the administration of an effective amount of beta=glycolipids, specifically. GC to subjects suffering from a neurodegenerative disorder or specifically, any of the protein-misfolding disorders disclosed herein, that are being treated, or previously treated with ambroxol.

Still further embodiments relate to methods of treating and preventing neurodegenerative disorders by administering an effective amount of ambroxol to subjects that are being treated, or previously treated with beta-glycolipides, specifically, GC.

It should be appreciated that the invention further encompasses methods that involve administration of an effective amount of at least one of ambroxol, GC or any combinations thereof to subjects that are being treated or previously treated with any drug that exhibit a neuroprotective effect, specifically any of the drugs disclosed by the invention.

In some embodiments, the method of the invention may further comprise an additional step of administering to the treated subject a therapeutically effective amount of at least one additional therapeutic drug. It should be appreciated that the additional therapeutic drug that in certain embodiments may be a drug having a neuroprotective effect, may be used for treating the indicated disorder, where the combination of the invention may be used as an additional and complementary treatment regimen.

It must be understood that the method of the invention encompasses the combined treatment discussed above administered in different dosage forms (e.g., oral and parenteral), at different dosage intervals. The two compounds of the combined treatment may be therefore administered either together, simultaneously, or alternatively, administered sequentially in either order.

In some further embodiments, the present invention provides methods for the treatment of neurodegenerative disorders in a subject in need thereof.

In certain embodiments, the methods of the invention may be particularly applicable for subjects suffering from a disorder associated with alpha synuclein pathology.

In yet some further embodiments, the methods of the invention may be applicable for treating alpha-synuclein pathology disorders that include at least one of PD. Dementia with Lewy Bodies (LB), MSA or any conditions, and/or any early signs or symptoms associated therewith.

In some specific embodiments, the method of the invention may be applicable for treating, preventing, reducing attenuating, inhibiting and eliminating PD, or any disorder, or any early signs or symptoms associated therewith, specifically, any dementia or cognitive decline.

“Parkinson's disease (PD)” as used herein, is a neurodegenerative disease resulting from degeneration of midbrain dopamine neurons and accumulation of alpha-synuclein containing Lewy bodies in surviving neurons. The diagnosis of PD is based on the presence of cardinal motor features in the absence of other aetiological conditions. These motor features include the classical triad of bradykinesia, a resting pill-rolling tremor, and rigidity typically in association with hypomimia, hypophonia, micrographia and postural instability. However, it is now recognized that not all patients exhibit these features in equal measure and that many nonmotor features occur in PD from the time of diagnosis. Indeed, there is now great interest in the idea that the nonmotor features of PD may even precede its diagnosis, constituting prodromal or premotor PD. These premotor features include problems with olfaction, constipation, mood and sleep, and following the clinical diagnosis of PD, they can become more prominent. Cognitive problems and dementia also commonly develop in PD, affecting almost 50% by 10 years from diagnosis. However, in some individuals with an alpha synucleinopathy, significant cognitive problems precede the onset of parkinsonian motor symptoms, and these cases are clinically classified with a diagnosis of Dementia with Lewy Bodies.

There is clearly a major degree of overlap between these two conditions both clinically and pathologically, but at present, the clinical distinction rests on the time interval between the onset of motor symptoms and dementia, with a minimum one year interval being required for a diagnosis of PD as opposed to Lewy body dementia (DLB). Multiple system atrophy (MSA) is the rarest of the three major alpha synucleinopathies and differs significantly from PD and DLB in terms of its clinical presentation and its more aggressive course, reflecting differences in the underlying neuroanatomical pathways involved.

The discovery that alpha synuclein was the major protein in Lewy bodies led to a re-evaluation of the pathology of PD at the end of the last century using immunohistochemical staining against this protein. This and led to a new pathological staging system for PD, in which the early stages were characterized by non-nigral pathology and the substantia nigra was affected only at stage 3. Stages 1 and 2 involved more caudal brainstem nuclei including the dorsal vagal nucleus and the locus coeruleus as well as the olfactory bulb. If this proposed pathological staging of PD was correct, then, this should have clinical correlations—i.e. patients at the time of presentation with their motor symptoms should have a prior history of anosmia, gut disturbances and changes in mood and sleep. Subsequently, this has been shown to be true for the majority of patients, although in clinical practice, these features are rarely volunteered by the patient who typically considers them irrelevant to their motor problems, and they often have to be specifically asked about. Thus, in the patient presenting for the first time, there is often a history of a loss of the sense of smell for some years constipation, anxiety or depression (although the hypomimia of PD can be mistaken for depression) and REM sleep behavioral disorder (RBD) in which patients act out their dreams.

In addition to the above prodromal PD, the clinical diagnosis of the disease includes three steps:

The first step includes the presence of bradykinesia with rigidity, rest tremor or postural instability.

The second step aims to trace red flags that raise the possibility of alternative diagnosis. These include repeated strokes with stepwise progression of parkinsonism, history of repeated head injury, history of definite encephalitis, oculogyric crises, neuroleptic treatment at symptom onset, sustained remission of symptoms, strictly unilateral features after three years, supranuclear gaze palsy, cerebellar signs, early severe autonomic involvement, early severe dementia with disturbances of memory, language, and praxis, babinski sign, presence of cerebral tumor or communicating hydrocephalus on brain imaging, negative response to large doses of levodopa in the absence of malabsorption. The third step includes supportive criteria for PD diagnosis: unilateral onset, presence of rest tremor, progressive disorder and persistent asymmetry.

While all of these symptoms can occur for a number of other reasons, their presence in a patient with subtle motor features of parkinsonism is very suggestive of a diagnosis of PD, although can also be seen in other alpha synucleinopathies.

The recognition of ‘premotor’ state of PD has had two major consequences to the field, in addition to helping diagnostically at the initial consultation. Firstly, it has led to clinical studies searching for even earlier pathology in structures projecting into the dorsal vagal nucleus, namely in the gut. This has revealed that the alpha synuclein pathology is present in the gastrointestinal tract not only in patients with established PD but in some cases in advance of the motor presentation. This supports the concept that PD has non-CNS pathology, and the disease may even begin outside the brain.

The second major consequence is that programmers of research have been set up to either screen ageing populations to identify individuals at risk of developing PD based on olfaction tests, sleep assessments dopamine scans or alternatively to follow-up individuals thought to be at high risk of developing an alpha synucleinopathy.

While the motor features of PD already described are the best recognized aspects of the condition, patients often have a range of non-motor symptoms in addition to those that characterize the prodromal phase. These include insomnia, fatigue, daytime somnolence, pain, urinary symptoms, sweating, mood and cognitive deficits. Some of these are in part secondary to the underlying motor problems, for example, the inability to move quickly and fluently will make one more tired, and difficulty moving at night will wake patients up and this can lead to the daytime symptoms of sleepiness, fatigue and a loss of motivation. Furthermore, the condition is very heterogeneous with patients presenting with different combinations of motor and nonmotor features, which makes generalizations about this condition difficult.

The nonmotor features are now considered to be a common feature of PD. These may reflect variation in the extent of alpha synuclein pathology in different cases, but also the extent of other pathological changes including Alzheimer's type changes, which are thought to contribute to the cognitive decline in PD. However, not all this variability in nonmotor features relates directly to underlying neurodegenerative pathology as nonmotor symptoms are exacerbated by the drugs used to treat PD, for example, dopamine agonists can cause daytime somnolence.

In the case of a tremor-dominant presentation, dopamine transporter SPECT scanning can be very helpful to distinguish PD (with striatal presynaptic dopamine deficiency) from other disorders, although, such scans cannot distinguish between PD and other neurodegenerative causes of parkinsonism such as MSA.

The clinical course that patients follow from the time of diagnosis is very variable. The average age of PD diagnosis is around 68 years of age. PD does not adversely affect life expectancy. Cause of death is not directly attributable to PD in the majority. The cumulative probability of developing PD with dementia (PDD) at 10 years into the illness is 46%. Almost 70% of patients will develop postural instability at 10 years, which is a key milestone in this condition. Differences in rate of progression to key milestones such as postural instability and PDD is presumed to reflect differences in the speed at which alpha-synuclein-related pathology spreads from relatively localized nigrostriatal involvement to widespread pathology throughout the cortex and brainstem, when significant gait and balance problems and dementia occur.

The commonest genetic factor associated with developing PD is mutations within GBA, the gene which encodes the enzyme glucocerebrosidase (GBA), which in their homozygous or compound heterozygous mutant form cause Gaucher's disease (GD). However, heterozygotes are predisposed to PD that typically presents slightly earlier in life and follows a more aggressive course than idiopathic PD. Patients in possession of such a mutation had a five times greater risk of dementia over a median follow-up period of 6 years from diagnosis. The mechanism by which defects in this gene cause disease is unclear as they do not seem to simply relate to decreased enzyme activity, but rather to abnormalities in lysosomal function. This may involve a positive feedback loop occurring once the disease starts activity and lysosomal function which in turn causes worsening alpha-synuclein aggregation and so on.

Another major genetic factor implicated in disease progression in PD is MAPT (microtubule-associated protein tau) haplotype, with the H1 haplotype being associated with an increased risk of developing the disease, and an earlier onset of PDD. The underlying mechanism is unclear, but possession of the H1 MAPT haplotype in the context of PD does seem to alter the ratio of 3-4 repeat tau which may be significant, given that tau pathology is reported to contribute to the dementia of PD in some cases Furthermore, tau aggregation may promote aggregation of alphasynuclein, with hybrid oligomers recently being reported in PD brain.

The motor course of PD, also termed as Motor progression, often follows a predictable course with patients initially responding well to dopaminergic medication for a number of years. The first sign that the treatment is starting to fail is that patients report that the effect of their L-dopa does not last until their next dose—the so-called “wearing off” phenomenon. This can be managed by adding in monoamine oxidase inhibitors or COMT inhibitors, but this subsequently gives way to more marked ‘on-off’ motor fluctuations with some doses of medication not producing any benefit. Patients also begin to experience involuntary movement when on their L-dopa, so-called L-dopa-induced dyskinesias (LIDs). These are exacerbated by stress, infections or tiredness. Initially, the patient is often oblivious to their existence, and they are noticed only by the spouse or carer, but ultimately they become more apparent to the patient. While dyskinesias can be improved by reducing the dose of L-dopa, patients generally prefer to maintain their L-dopa dose in spite of these movements rather than reduce it and increase their time in an ‘off’ state, which most patients find to be one of the most frightening parts of their illness. The rate at which dyskinesias develop is dependent on the age of onset and the dose of L-dopa they are taking, but most patients will have some LIDs by 10 years from diagnosis, with an estimated mean time to dyskinesia onset of 6.6 years. They are initially managed by fractionating the L-dopa medication into more frequent doses, and introducing amantadine. As the dyskinesias become more troublesome, management options include the introduction of continuous dopaminergic therapies such as apomorphine or enteral DuoDopa pumps, or deep brain stimulation, typically to the subthalamic nucleus, which allows for significant reduction in L-dopa dosing. In addition to these motor fluctuations, patients typically experience increasing problems with their gait in the later stages of their disease due to freezing of gait, balance problems and postural hypotension related to autonomic disturbance as well as medication. Indeed, clinically there seems to a be a critical tipping point in patients when they develop a triad of problems with postural hypotension, freezing of gait and neuropsychiatric problems, and at this stage, it seems that the alpha synuclein pathology has spread to involve areas where dopaminergic drugs not only do not help, but can actually make things worse. Such a state is often the harbinger of the final stages of PD and the need to start planning long-term care packages and nursing home placement.

The development of neuropsychiatric features is more variable with respect to PDD. The most common neuropsychiatric problems relate to mood disturbance, with depression affecting around 35% of PD patients. Depression is thought to be an intrinsic feature of the condition related to disturbances of noradrenergic, serotonergic and dopaminergic transmission as well as Lewy body deposition in limbic structures. In some cases, depression in PD can be severe and resistant to treatment with conventional anti-depressant therapies, and Electroconvulsive therapy (ECT) is needed. However, in most cases, it is relatively mild. Anxiety may also affect up to a third of PD patients, either co-existing with depression or occurring independently. It is often seen in the context of tremor-dominant disease, and can also be particularly problematic during ‘off’ fluctuations. With disease progression, other neuropsychiatric problems emerge and are often a harbinger of PDD. They are typically aggravated by the dopaminergic medication, and by the amantadine used to manage the LIDs. These problems initially take the form of visual phenomena, with the patient thinking they can see something out of the corner of their eye, although when they turnaround to see what it is, it is not there. When present, it is easy to see how this can lead to a degree of paranoia, with patients experiencing a feeling that they are being spied on. In addition, they also start to experience hallucinations, typically in the visual domain. This involves seeing animate objects, often at times of low illumination and driven by misperception of real objects, such as complex patterns on carpets or objects sitting on top of cupboards. These creatures are initially recognized as not being real, but with time this insight is lost and visual hallucinations can take the form of grotesque shapes as well as people. These symptoms are often very distressing and can lead to significant behavioural problems, which necessitate reducing their standard medication and/or introducing treatment with cholinesterase inhibitors or low dose quetiapine. In some patients, these problems can develop into a whole delusional system.

Although the development of these features can be controlled pharmacologically in their early stages, they will tend to recur and progress and their presence typically suggests that the patient is beginning to develop PDD with accompanying cortical Lewy body pathology.

The other major developments in advancing PD involve problems with speech, swallowing, the gut and the autonomic nervous system. Speech becomes very quiet and slurred, and in the advanced stages, it can be very hard to understand what is being said, which can generate levels of frustration. It rarely responds to dopaminergic therapy or speech and language therapy at the more advanced stages, and may even be made worse by Deep Brain Stimulation (DBS). It can though occasionally respond to such interventions when given earlier in the condition. Swallowing problems can develop in later disease, which can be particularly problematic as patients with PD also tend to lose weight. In advanced cases, enteral feeding through a Percutaneous Endoscopic Gastroscopy (PEG) can be considered.

Most patients with PD will have some degree of autonomic involvement with the most common manifestation of this being postural hypotension, which again can be made worse by some of the medications used to treat the motor features of this condition. It can be subclinical in some cases, and in the absence of symptoms, requires no treatment. In more advanced stages, it can cause blackouts (which can lead to other problems such as fractured hips and wrists or subdural haematomas) and needs to be treated with either fludrocortisone, domperidone, midodrine or a combination thereof in severe cases. Other autonomic features in PD include problems with excessive sweating, bladder irritability and constipation. This latter feature is found in nearly all cases of PD, and as previously discussed, may even precede the development of motor features of the condition. The cause of the constipation in part relates to lack of mobility, but may also be due to alpha synuclein pathology in the enteric and autonomic nervous system.

Sleep problems are common in PD. In addition to the RBD which can occur from the prodromal phase, many patients have overnight motor problems, which can lead to disturbed sleep including restless leg syndrome, and akinesia related to being ‘off’. In addition, some patients experience vivid dreams with L-dopa medications and this too can disrupt sleep. Recent evidence also suggests that there may be an intrinsic circadian rhythm abnormality from early stages of PD with altered cortisol and melatonin levels, as well as changes in peripheral clock gene expression. Finally, patients can experience other problems such as difficulty with opening their eyes (eyelid opening apraxia), cramps, pain and sensory symptoms many of which relate to the underlying motor state.

It should be therefore appreciated that the combinations, compositions, kits and methods of the invention that offer combined treatment with bata-glycolipids, specifically, glucocerebroside and trans-4-(2-Amino-3,5-dibromobenzylamino) cyclohexanol hydrochloride, specifically ambroxol, are applicable for treating any of the stages, symptoms, clinical manifestations or conditions associated with PD, specifically as disclosed herein above.

In some specific embodiments, the method of the invention may be particularly applicable in prevention of PD in a subject in need thereof. In more specific embodiments, such subject may be a subject at risk for developing PD and/or a subject that displays early singes or symptoms associated with PD.

In more specific embodiments, a subject at risk for developing PD may be for example a subject that carry at least one mutation in at least one gene encoding glucocerebrosidase (GBA) or in a gene encoding any protein associated therewith, specifically any protein that is associated or connected with GBA stability, expression or activity.

It should be noted that Glucocerebrosidase (also called acid β-glucosidase, D-glucosyl-N-acylsphingosine glucohydrolase, or GCase) as used herein is an enzyme having glucosylceramidase activity that hydrolyze the beta-glucosidic linkage of glucocerebroside, an intermediate in glycolipid metabolism. It is localized in the lysosome and has a molecular weight of 59.7 KD. In yet some further specific embodiments, the Glucocerebrosidase as used herein refers to the human Glucocerebrosidase, specifically, the human Glucocerebrosidase that comprise the nucleic acid sequence as disclosed by GenBank accession number NM_000157, or any variant or homolog thereof.

In yet some further embodiments, a subject at risk in accordance with the invention may be a subject that carry at least one mutation in GCase gene. In more specific embodiments, such mutations may include but are not limited to L444P, 84CGG, N370S or any other mutation in the coding or the non-coding region of the gene. It should be further noted that the subject may be either homozygous or heterozygous for such mutation to L444P, 84GG, N370S.

In yet some further embodiments, a subject at risk may be a subject that suffers from GD. More specifically, Gaucher's disease or Gaucher disease (GD) is a genetic disorder in which glucocerebroside (a sphingolipid, also known as glucosylceramide) accumulates in cells and certain organs. The disorder is characterized by bruising, fatigue, anemia, low blood platelet count and enlargement of the liver and spleen, and is caused by a hereditary deficiency of the enzyme glucocerebrosidase. Manifestations may include enlarged spleen and liver, liver malfunction, skeletal disorders or bone lesions that may be painful, severe neurological complications, swelling of lymph nodes and adjacent joints, distended abdomen, a brownish tint to the skin, anemia, low blood platelet count, and yellow fatty deposits on the white of the eye (sclera). GD has three common clinical subtypes. GD type I (non-neuropathic) is the most common and least severe form of the disease. Symptoms may begin early in life or in adulthood and mainly affect the liver, spleen, and bone. Enlarged liver and grossly enlarged spleen (together hepatosplenomegaly) are common. The brain and nervous system are not affected pathologically, but lung and, rarely, kidney impairment may occur. GD type II (acute infantile neuropathic) typically begins within 6 months of birth and has an incidence rate around one 1 in 100,000 live births. Symptoms include an enlarged liver and spleen, extensive and progressive brain damage, eye movement disorders, spasticity, seizures, limb rigidity, and a poor ability to suck and swallow. Affected children usually die by age two. GD type III (chronic neuropathic) can begin at any time in childhood or even in adulthood, and occurs in about one in 100,000 live births. It is characterized by slowly progressive, but milder neurologic symptoms compared to the acute or type II version. Major symptoms include an enlarged spleen and/or liver, seizures, poor coordination, skeletal irregularities, eye movement disorders, blood disorders including anemia, and respiratory problems or any symptom which is directly or indirectly associated with the disease.

It should be appreciated that the method of the invention may be applicable for any stage, type, degree, phase, level of PD, or for any symptom or condition associated therewith.

In yet some further embodiments, the method of the invention may result in improvement in different parameters associated with PD. In non-limiting embodiments, such parameters may include at least one of: the mean change in the motor score (part II) of the unified Parkinson's disease rating scale (UPDRS score); Mean change in total UPDRS score (I-III); Montreal Cognitive Assessment (MoCA) score; Timed up-and-go test; Purdue pegboard; Neurotrax; The Patient Global Impression of Improvement (PGI-I); Parkinson's disease questionnaire (PDQ-39); Epworth Sleepiness Scale; Beck Depression Inventory; Frontal assessment battery (FAB); Addenbrooke's Cognitive Examination; Questionnaire for Impulsive-Compulsive Disorders in Parkinson's (QUIP-RS), Smell test; Substantia nigra (SN) ultra-sound hyperechogenicity (>0.2); Thinning of the retina measured by OCT; Lyso Gb1; Color discrimination test; and Orthostatic hypotension.

More specifically, the unified Parkinson's disease rating scale (UPDRS), is used to follow the longitudinal course of Parkinson's disease, and comprise the following sections: Part I: evaluation of mentation, behavior, and mood; Part II: self-evaluation of the activities of daily life (ADLs) including speech, swallowing, handwriting, dressing, hygiene, falling, salivating, turning in bed, walking, and cutting food; Part III: clinician-scored monitored motor evaluation; Part IV: complications of therapy; Part V: Hoehn and Yahr staging of severity of Parkinson's disease; and Part VI: Schwab and England ADL scale.

Montreal Cognitive Assessment (MoCA) score as used herein, is a test designed as a rapid screening instrument for mild cognitive dysfunction. It assesses different cognitive domains: attention and concentration, executive functions, memory, language, visuo-constructional skills, conceptual thinking, calculations, and orientation. Time to administer the MoCA is approximately 10 minutes. The total possible score is 30 points; a score of 26 or above is considered normal.

“Timed up-and-go test (TUG)” as used herein is a simple test used to assess a person's mobility and requires both static and dynamic balance. It uses the time that a person takes to rise from a chair, walk three meters, turn around, walk back to the chair, and sit down. During the test, the person is expected to wear the regular footwear and use any mobility aids that he/she would normally require. Scores of ten seconds or less indicate normal mobility, 11-20 seconds are within normal limits for frail elderly and disabled patients, and greater than 20 seconds means the person needs assistance outside and indicates further examination and intervention. A score of 30 seconds or more suggests that the person may be prone to falls. Alternatively, a recommended practical cut-off value for the TUG to indicate normal versus below normal performance is 12 seconds. TUG performance has been found to decrease significantly with mobility impairments. Residential status and physical mobility status have been determined to be significant predictors of TUG performance.

“The Purdue Pegboard Test” as used herein is a neuropsychological test of manual dexterity and bimanual coordination. The test involves two different abilities: gross movements of arms, hands, and fingers, and fine motor extremity, also called “fingerprint” dexterity. Poor Pegboard performance is a sign of deficits in complex, visually guided, or coordinated movements that are likely mediated by circuits involving the basal ganglia of a brain. The pegboard consists of a board with two parallel rows with 25 holes into which cylindrical metal pegs are placed by the examinee. The test involves a total of four trials. To begin, there is a brief practice. The subsets for preferred, non-preferred, and both hands require the patient to place the pins in the holes as quickly as possible, with the score being the number of pins placed in 30 seconds.

“NeuroTrax™ Computerized test” as used herein relates to assessment of brain wellness across an array of cognitive domains including: memory, executive function, visual spatial perception, verbal function, attention, information processing speed, and motor skills. The psychometric properties of the tests exploit the advantages of computerized testing, providing precise accuracy and reaction time measurements. NeuroTrax offers an unbiased, standardized, accurate and inexpensive tool with a wide range of applicability.

Participants are presented with 10 pairs of words to study followed by a recognition test in which they are presented with one member of a previously presented pair together with four possible alternatives for the other member of the pair. Responses are made using the keyboard number pad to indicate which pair was previously presented. Up to four consecutive study/test repetitions follow immediately, and an additional recognition test is administered following two other NeuroTrax tests for a delay period of approximately 10 minutes.

“The Patient Global Impression of Improvement (PGI-I)” test as used herein refers to a global index that may be used to rate the response of a condition to a therapy (transition scale). It is a simple, direct, easy to use scale that is intuitively understandable to clinicians. The PGI-I has only been tested on women with stress urinary incontinence. The PGI-I is a transition scale that is a single question asking the patient to rate their urinary tract condition now, as compared with how it was prior to before beginning treatment on a scale from 1 to 7.

The “39-item Parkinson's Disease Questionnaire”, and particularly its summary index (PDQ-39SI), as used herein, is a widely used patient-reported clinical trial endpoint. Substantial evidence is available to suggest that the PDQ is reliable, valid, responsive, acceptable and feasible as the tool for the assessment of quality of life in Parkinson's disease patients. For these reasons it has been widely adopted and generally considered the industry ‘gold standard’. The PDQ-39 is primarily used in clinical trials of therapeutics intended to benefit individuals with Parkinson's disease.

More specifically, there are 39 questions in the long form Parkinson's Disease Questionnaire, provides scores for each of the: mobility, activities of daily living, emotional well-being, stigma, social support, cognitions, communications and bodily discomfort. Alternatively, the sum of the scores can provide a single figure used to assess the overall health-related quality of life profile of the individual questioned. Details of the scoring system and administration procedures for PDQ-39 and PDQ-8 are provided in the PDQ User Manual. A short form of the PDQ-39, the PDQ-8 has been developed using questions taken from the larger instrument to provide a single index score that is almost identical to those gained from the PDQ-39. It should be noted that the invention further encompasses the use of this shortened version.

“Epworth Sleepiness Scale” as used herein is a scale intended to measure daytime sleepiness that is measured by use of a very short questionnaire. This can be helpful in diagnosing sleep disorders. The questionnaire asks the subject to rate his or her probability of falling asleep on a scale of increasing probability from 0 to 3 for eight different situations that most people engage in during their daily lives, though not necessarily every day. The scores for the eight questions are added together to obtain a single number. A number in the 0-9 range is considered to be normal while a number in the 10-24 range indicates that expert medical advice should be sought. For instance, scores of 11-15 are shown to indicate the possibility of mild to moderate sleep apnea, where a score of 16 and above indicates the possibility of severe sleep apnea or narcolepsy.

“Beck Depression Inventory (BDI)” as used herein relates to a 21-question multiple-choice self-report inventory, one of the most widely used psychometric tests for measuring the severity of depression. Its development marked a shift among mental health professionals, who had until then, viewed depression from a psychodynamic perspective, instead of it being rooted in the patient's own thoughts. In its current version, the BDI is designed for individuals aged 13 and over, and is composed of items relating to symptoms of depression such as hopelessness and irritability, cognitions such as guilt or feelings of being punished, as well as physical symptoms such as fatigue, weight loss. The BDI is widely used as an assessment tool by health care professionals and researchers in a variety of settings. When the test is scored, a value of 0 to 3 is assigned for each answer and then the total score is compared to a key to determine the depression's severity. The standard cut-off scores are as follows: 0-9: indicates minimal depression: 10-18: indicates mild depression; 19-29: indicates moderate depression and 30-63: indicates severe depression.

“Frontal assessment battery (FAB)” as used herein is a short cognitive and behavioural six-subtest battery for the bedside screening of a global executive dysfunction; although recently devised, it is already extensively used thanks to its ease of administration and claimed sensitivity. The purpose of the FAB is in discriminating between dementias with a frontal dysexecutive phenotype and Dementia of Alzheimers Type (DAT). The FAB has validity in distinguishing Fronto-temporal type dementia from DAT in mildly demented patients. Total score is from a maximum of 18, higher scores indicating better performance.

“Addenbrooke's Cognitive Eramination” as used herein relates to a simple bedside test battery designed to detect mild dementia and differentiate Alzheimer's Dementia (AD) from frontotemporal dementia (FTD). Addenbrooke's Cognitive Examination (ACE) is a 100-point test battery that assesses six cognitive domains. The composite and the component scores on the ACE for the patients' groups are compared with age- and education-matched controls. Norms and the probability of diagnosing dementia at different prevalence rates are calculated. To evaluate the ACE's ability to differentiate early AD from FTD, scores of the cases diagnosed with dementia with a Clinical Dementia Rating <1 (AD=56, FTD=24, others=20) are compared.

“Questionnaire for Impulsive-Compulsive Disorders in Parkinson's disease rating scale (QUIP-RS)” as used herein is appropriate for assessment of Impulsive-Compulsive Disorders in Parkinson's disease (PD). A rating scale designed to measure severity of symptoms and supports a diagnosis of impulse control disorders and related disorders in PD. A PD patient self-complete the QUIP-RS and is administered a semistructured diagnostic interview by a blinded trained rater to assess discriminant validity for impulse control disorders and related disorders. The QUIP-RS appears to be valid and reliable as a rating scale for impulse control disorders and related disorders in PD. The test can be used to support a diagnosis of these disorders, as well as to monitor changes in symptom severity over time.

“Smell test between weeks 0 and 72” as used herein refers to the University of Pennsylvania Smell Identification Test (UPSIT) that is commercially available for smell identification to test the function of an individual's olfactory system. It is the gold standard of smell identification tests for its reliability (r=0.94) and practicality. This test has been used as a test in the diagnosis of many diseases including Parkinson's Disease and Alzheimer's. This test is a measurement of the individual's ability to detect odors at a suprathreshold level. The test is usually administered in a waiting room and takes only a few minutes. The test consists of 4 different 10 page booklets, with a total of 40 questions. On each page, there is a different “scratch and sniff” strip which are embedded with a microencapsulated odorant. There is also a four choice multiple choice question on each page. The scents are released using a pencil. After each scent is released, the patient smells the level and detects the odor from the four choices. There is an answer column on the back of the test booklet, and the test is scored out of 40 items. The score is compared to scores in a normative database from 4000 normal individuals.

“Substantia nigra (SN) ultra-sound hyperechogenicity (>0.2)” as used herein reters to the detection of an enlarged echogenic size (“hyperechogenicity”) of the substantia nigra on transcranial sonography for the early and differential diagnosis of Parkinson disease. This examination is accompanyed with video and presents assessment of the substantia nigra, planimetric measurement of substantia nigra echogenicity according to current guidelines, for diagnostic implications in patients with parkinsonism.

“Color discrimination test” as used herein is employed to test the ability of patients with Parkinson's disease to discriminate colors, since the impairment of color discrimination may be an early sign in Parkinson's disease. The term for describing a lack of perceptual sensitivity to certain colors, is Color Vision Deficiency (CVD). The most commonly used test to detect color vision deficiencies consists of plates filled with colored dots. The dots are colored in different shades and a number is hidden inside with shades of another color.

“Thinning of the retina measured by OCT” as used herein refers to the investigation of the changes of retinal thickness in macula of eyes using spectral domain optical coherence tomography (OCT). The possible retinal structural changes in individuals can be diagnosed by measuring the retinal thickness in the macular region using spectral domain OCT under free living conditions.

“Lyso Gb1” as used herein refers to the use of primary storage molecules such as glucosylceramide (Gb1) as biomarker in plasma of Gaucher's disease patients and to compare its levels to the level of Gb1 in healthy individuals. Lyso-Gb1 concentration shows correlation and has a predictive value concerning the clinical severity of Gaucher's disease.

The term “Orthostatic hypotension (OH)” as used herein, relates to a condition when mechanisms for the regulation of orthostatic (while standing) blood pressure control fails. Such regulation depends on the baroreflexes, normal blood volume, and defenses against excessive venous pooling. Orthostatic hypotension refers to the development of symptoms such as lightheadedness and blurred vision when a subject stands up that clears on sitting back down. Other symptoms include cognitive blunting, tiredness, and head and neck ache. These symptoms are due to cerebral hypoperfusion. OH is common in the elderly and is associated with an increase in mortality rate. There are many causes of OH. Aging coupled with diseases such as diabetes and Parkinson's disease results in a prevalence of 10-30% in the elderly. These conditions cause baroreflex failure with resulting combination of OH, supine hypertension, and loss of diurnal variation of blood pressure. The goal of the treatment is to improve standing blood pressure so as to minimize symptoms and to improve standing time in order to be able to undertake orthostatic activities of daily living, without excessive supine hypertension. Therefore OH is assessed before and after the treatment.

As noted above, in some embodiments, the method of the invention may result in or lead to improvement of any of the disclosed parameters or any manifestation thereof, in the treated subject. The term “improvement” as used herein relates to advancement, upgrade, refinement, amelioration or change for better of any of the symptoms or the examined parameters, scores disclosed herein in at least about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, abolt 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or 100%.

In yet some other specific embodiments, the present invention provides a method for treating, preventing, reducing, attenuating, inhibiting and eliminating a disorder associated with Dementia with Lewy Bodies (DLB) and/or any early signs or symptoms associated therewith

“Dementia with Lewy Bodies (DLB)”, as used herein, is a relatively common cause of dementia, estimated to account for up to 30% of dementia cases, and affecting up to 5% of those over the age of 75. Pathologically, it is defined by the presence of alpha synuclein containing Lewy bodies in the brain, but their distribution differs from that in PD, affecting the neocortex, limbic system and brainstem, in contrast to the nigrostriatal and brainstem-predominant pattern seen in early PD. However, as previously discussed, in the later stages of PD, cortical Lewy body pathology is common, along with development of a dementia, and it is debated whether DLB and PDD essentially represent the same pathological process separated by an arbitrary time factor or whether in fact they are truly different conditions. Recent genetic studies looking at variation within the alpha synuclein gene and risk of PD, PDD and DLB have indicated that these disorders have distinct genetic association profiles suggesting that they are in fact different disease processes with a common pathology. Imaging studies have also indicated that DLB and PDD differ with respect to the cortical amyloid load, which is much greater in DLB and may be an important contributor to the early cognitive impairment in this condition. Clinically, DLB is characterized by a progressive dementia with prominent visual hallucinations and delusions, and parkinsonism with bradykinesia and rigidity but typically minimal tremor. Marked cognitive fluctuations are a common feature of this condition, with episodes of confusion, excessive somnolence, and incoherent speech which can revert to a near normal state within hours. In all these respects, it can look very similar to PDD. The diagnostic distinction between the two conditions rests mainly in the difference in the temporal evolution of symptoms, with DLB being diagnosed in patients who develop a dementia within a year of developing parkinsonian features, while PDD defines people with pre-existing PD who then go on to develop a dementia at least one year after motor disease onset. Both DLB and PDD can be treated with cholinesterase inhibitors, which have some efficacy in the earlier stages of the dementia, and can be helpful for the hallucinations and behavioural symptoms as well as cognitive impairment. However, eventually the disease fails to respond to any treatment and palliative care is all that can be offered. It should be appreciated that the method of the invention may be applicable for any stage, type, degree, phase, level of DLB, or for any symptom or condition associated therewith.

In yet some more specific embodiments, the present invention provides a method for treating, preventing, reducing, attenuating, inhibiting and eliminating a disorder associated with MSA.

“Multiple system atrophy (MSA)”, as used herein, is much rarer than PD with an estimated prevalence of 4.4 per 100 000 (PD is around 45 times more common). It also tends to present earlier than PD, with a mean age of onset of 54 years. The neuropathology includes cell loss and gliosis in nigrostriatal and olivopontocerebellar structures, and the pathological signature of this condition differs from the Lewy bodies of PD, and instead takes the form of glial cytoplasmic inclusions containing fibrillar alpha-synuclein within oligodendrocytes. It presents with autonomic dysfunction along with parkinsonism and cerebellar dysfunction in varying combinations, and is clinically classified as being either mainly cerebellar in its presentation (MSA-C) or mainly parkinsonian (MSA-P). It typically follows a more aggressive course than PD with minimal response to dopaminergic medications. MRI can be helpful in some cases in distinguishing it from PD through the presence of a ‘hot cross bun’ sign in the pons or T2 signal changes in the dorsolateral putamen. MSA-C presents with a late-onset cerebellar syndrome causing ataxia and dysarthria, with evidence of involvement of other systems such as autonomic disturbances (impotence, bladder dysfunction, postural hypotension), a degree of parkinsonism, and/or pyramidal signs such as brisk reflexes and spasticity in the limbs. In contrast, MSA-P presents with a predominant parkinsonian syndrome which is often mistaken for PD, but with early prominent autonomic problems, a poor response to dopaminergic therapy, a gait that is often worse than anticipated from examination on the bed (because of the subtle cerebellar problems), a history of RBD and ‘sighing’ with a degree of inspiratory stridor. These latter signs and symptoms need to specifically be asked and looked for to aid the diagnosis.

Significant cognitive dysfunction is not a prominent feature of MSA, but cognitive deficits can occur in this condition, predominantly in the frontal executive domain. It should be appreciated that the method of the invention may be applicable for any stage, type, degree, phase, level of MSA, or for any symptom or condition associated therewith.

Multiple system atrophy evolves much more rapidly than PD, and patients soon become severely disabled with frequent falls, significant autonomic problems and difficulty with swallowing and speech. Their life expectancy is significantly shortened, with a median survival of about 6 years, but this is shorter in those with an older age at diagnosis.

There are no major therapeutic interventions that improve this condition other than supportive therapies.

The present invention further provides methods for treatment of neurodegenerative disorders, specifically disorders characterized by beta-amyloid protein aggregation, for example, Alzheimer's disease. The invention further provides methods for treating neurodegenerative disorders associated with age, specifically, age-related cognitive decline (ACD) and mild cognitive impairment (MCI). Such diseases are currently incurable, resulting in progressive degeneration and/or death of neuron cells.

As indicated above, in some embodiments, the invention provides methods for treating Alzheimer's disease.

“Alzheimer's disease (AD)”, as used herein refers to a disorder that involves deterioration of memory and other cognitive domains that leads to death within 3 to 9 years after diagnosis. The principal risk factor for Alzheimer's disease is age. The incidence of the disease doubles every 5 years after 65 years of age, however, up to 5% of people with the disease have early onset AD (also known as younger-onset), that may appear at 40 or 50 years of age.

Many molecular lesions have been detected in Alzheimer's disease, but the overarching theme to emerge from the data is that an accumulation of misfolded proteins in the aging brain results in oxidative and inflammatory damage, which in turn leads to energy failure and synaptic dysfunction.

Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia. Aging itself causes synaptic loss, which particularly affects the dentate region of the hippocampus.

Disruptions of the release of presynaptic neurotransmitters and postsynaptic glutamatereceptor ion currents occur partially as a result of endocytosis of N-methyl-D-aspartate (NMDA) surface receptors and endocytosis of α-amino-3-hydroxy-5-methyl-4-isoxazole propionic acid surface receptors. The latter further weakens synaptic activity by inducing a lasting reduction in currents after a high-frequency stimulus train. A similar shift in the balance between potentiation and depression in synapses occurs with normal aging. Intraneuronal Aβ can trigger these synaptic deficits even earlier. The normally high levels of neurotrophin receptors in cholinergic neurons in the basal forebrain are severely reduced in late-stage Alzheimer's disease. Aβ is a potent mitochondrial poison, especially affecting the synaptic pool. In Alzheimer's disease, exposure to Aβ inhibits key mitochondrial enzymes in the brain and in isolated mitochondria. Cytochrome c oxidase is specifically attacked. Consequently, electron transport, ATP production, oxygen consumption, and mitochondrial membrane potential all become impaired. The accumulation of Aβ within structurally damaged mitochondria isolated from the brains of patients with Alzheimer's disease.

There is no single linear chain of events or pathways that could initiate and drive Alzheimer's disease. AD is a progressive disease, where dementia symptoms gradually worsen over a number of years. In its early stages, memory loss is mild, but with late-stage AD, individuals lose the ability to carry on a conversation and respond to their environment. Those with AD live an average of eight years after their symptoms become noticeable to others, but survival can range from four to 20 years, depending on age and other health conditions.

The most common early symptom of AD is difficulty remembering newly learned information because AD changes typically begin in the part of the brain that affects learning. As AD advances through the brain it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

Beside symptomatic treatments to temporarily slow the worsening of dementia symptoms, AD has no current cure, and the current treatments cannot stop AD from progressing.

It should be appreciated that the combinations, compositions, kits and methods of the invention are suitable for treating any stage of AD, at any age and for any conditions and symptoms associated therewith.

As indicated above, plaques and tangles are involved with AD as well as in other age-related neurodegenerative processes. Thus, it should be appreciated that the invention further encompasses the use of the combined therapy disclosed herein for treating other age-related conditions.

With an increasingly aged population, cognitive impairment is a major health and social issue. Cognitive decline is among the most feared aspects of growing old. It is also the most costly, in terms of the financial, personal and societal burdens. It is important, because cognitive decline heralds dementia, illness and death.

The invention therefore in certain embodiments thereof, provides methods for treating, preventing, inhibiting, reducing, eliminating, protecting or delaying the onset of age-associated mild cognitive impairment (MCI).

“Age-associated mild cognitive impairment (MCI)”, as use herein is a condition that causes cognitive changes. MCI that primarily affects memory may be classified as “amnestic MCI” where the subjects experience impairment in memorizing information that relate to recent events, appointments or conversations or recent events. MCI that affects thinking skills other than memory is known as “nonamnestic MCI”. Thinking skills that may be affected by nonamnestic MCI include the ability to make sound decisions, judge the time or sequence of steps needed to complete a complex task, or visual perception.

Normal aging is associated with a decline in various memory abilities in many cognitive tasks; the phenomenon is known as age-related memory impairment (AMI), age-associated memory impairment (AAMI) or age-associated cognitive decline (ACD). The ability to encode new memories of events or facts and working memory shows decline in both cross-sectional and longitudinal studies. Studies comparing the effects of aging on episodic memory, semantic memory, short-term memory and priming find that episodic memory is especially impaired in normal aging; some types of short-term memory are also impaired. The deficits may be related to impairments seen in the ability to refresh recently processed information.

Normally, there is little age-associated decline in some mental functions such as verbal ability, some numerical abilities and general knowledge but other mental capabilities decline from middle age onwards, or even earlier. The latter include aspects of memory, executive functions, processing speed and reasoning. It should be therefore appreciated that in some embodiments, the invention provides combined treatment for any cognitive decline, specifically cognitive decline associated with age, specifically, the age of 50, 55, 60, 65, 70, 75, 80, 85, 90, 95 and more, years of age.

As disclosed herein above, neurodegeneration is the umbrella term for the progressive loss of structure or function of neurons, including death of neurons. Many neurodegenerative diseases including Parkinson's. Alzheimer's, ALS and Huntington's occur as a result of neurodegenerative processes. Other examples of neurodegeneration include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidoses and Charcot Marie Tooth. It should not be overlooked that normal aging processes include progressive neurodegeneration.

Still further, it should be appreciated that the invention provides methods for treating or preventing any neuro-pathological condition. The term “neuro-pathological condition” relates to any pathological condition caused by, or which causes, or is associated with neural cell disorders, such as any deterioration of the neural cell functions or viability. As explained herein, such conditions may be neurodegenerative disorders, ischemic diseases, brain traumas, metabolic disorders which affect the nervous system, such as diabetes and phenylketonuria, immunological disorders which affect the brain, such as Hashimoto's Thyroiditis, genetic diseases which affects neural cells, such as Tay-Sachs disease, metachromatic leukodystrophy. Krabbe disease. Fabry disease, Gaucher disease, Farber disease, and Niemann-Pick disease, nutrient deficiencies such as vitamin B₆ and D deficiencies, and any sequelae which affects the nervous system.

It should be further appreciated that the combined kits, methods and compositions of the invention may be applicable for treating neuro-pathological and neurodegenerative disorders or of any pathologic condition associated therewith. It is understood that the interchangeably used terms “associated”, linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. Such conditions may include for example, Parkinson's disease, Alzheimer's disease, amyotrophic lateral sclerosis, head trauma, epilepsy, stroke, neuromyotonia/Isaacs syndrome, lower motor neuron lesion, Werdnig-Hoffman disease, amyotrophic lateral sclerosis, Kennedy disease, organophosphate poisoning, benzodiazepine withdrawal, magnesium deficiency, myalgic encephalomyelitis, dehydration, fatigue, lyme disease, myasthenia gravis, rabies, fibromyalgia, subarachnoid hemorrhage, intracerebral hemorrhage, occlusion and stenosis of precerebral arteries, occlusion and stenosis of basilar artery, occlusion and stenosis of carotid artery, occlusion and stenosis of vertebral artery, occlusion of cerebral arteries, cerebral thrombosis with or without cerebral infarction, cerebral embolism with or without cerebral infarction, transient cerebral ischemia, basilar artery syndrome, vertebral artery syndrome, subclavian steal syndrome, vertebrobasilar artery syndrome, transient ischemic attack (TIA), cerebral atherosclerosis, hypertensive encephalopathy, cerebral aneurysm, cerebral arteritis. Moyamoya Disease, nonpyogenic thrombosis of intracranial venous sinus, atherosclerosis, atherosclerosis of renal artery, atherosclerosis of native arteries of the extremities, intermittent claudication, aortic aneurysm, dissection of aorta, dissection of carotid artery, dissection of iliac artery, dissection of renal artery, dissection of vertebral artery, erythromelalgia, and polyarteritis nodosa.

It is appreciated that the invention is also applicable to neurodegenerative disorders that are metabolic (diabetes) related, and toxin-induced.

In yet some other embodiments, the method of the invention may be applicable in treating condition involving neurological injury. The term “condition involving neurological injury” refers to traumatic head or brain injury (including epilepsy), spinal cord injury, peripheral nerve injury or peripheral neural cell injury.

Still further, in certain embodiments, the method of the invention may be used for treating ALS. Amyotrophic lateral sclerosis (abbreviated ALS, also referred to as Lou Gehrig's disease) is a form of motor neuron disease caused by the degeneration of neurons located in the ventral horn of the spinal cord and the cortical neurons that provide their afferent input. The disorder is characterized by rapidly progressive weakness, muscle atrophy and fasciculations, spasticity, dysarthria, dysphagia, and respiratory compromise. Sensory function generally is spared, as is autonomic, and oculomotor activity. ALS is a progressive, fatal, neurodegenerative disease with most affected patients dying of respiratory compromise and pneumonia after 2 to 3 years; although occasional individuals have a more indolent course and survive for many years. Another embodiment of the invention provides a method for treating HD. Huntington's disease (HD) causes astrogliosis and loss of medium spiny neurons. Areas of the brain are affected according to their structure and the types of neurons they contain, reducing in size as they cumulatively lose cells. The areas affected are mainly in the striatum, but also the frontal and temporal cortices. The striatum's subthalamic nuclei send control signals to the globus pallidus, which initiates and modulates motion. The weaker signals from subthalamic nuclei thus cause reduced initiation and modulation of movement, resulting in the characteristic movements of the disorder. Mutant Huntingtin is an aggregate-prone protein.

As noted above, the greatest risk factor for neurodegenerative diseases is aging. Mitochondrial DNA mutations as well as oxidative stress both contribute to aging. Many of these diseases are late-onset, meaning there is some factor that changes as a person ages for each disease. One constant factor is that in each disease, neurons gradually lose function as the disease progresses with age. Therefore, according to certain embodiments, the methods of the invention are applicable for treating ischemic disease or condition. In a more specific embodiment, the ischemic disease or condition is stroke.

A stroke, previously known medically as a cerebrovascular accident (CVA), is the rapidly developing loss of brain function(s) due to disturbance in the bloxxod supply to the brain. This can be due to ischemia (lack of blood flow) caused by blockage (thrombosis, arterial embolism), or a hemorrhage (leakage of blood). As a result, the affected area of the brain is unable to function, which might result in an inability to move one or more limbs on one side of the body, inability to understand or formulate speech, or an inability to see one side of the visual field. A stroke is a medical emergency and can cause permanent neurological damage, complications, and death. It is the leading cause of adult disability in the United States and Europe and the second leading cause of death worldwide. Risk factors for stroke include old age, hypertension (high blood pressure), previous stroke or transient ischemic attack (TIA), diabetes, high cholesterol, cigarette smoking and atrial fibrillation. High blood pressure is the most important modifiable risk factor of stroke.

In yet another embodiment, the method of the invention may be used for treating a condition involving neuronal injury. In another specific embodiment, the condition involving neurological injury may be any one of acute, traumatic or chronic brain injury.

An acute brain injury or traumatic brain injury (TBI) is a nondegenerative, noncongenital insult to the brain from an external mechanical force, possibly leading to permanent or temporary impairment of cognitive, physical, and psychosocial functions, with an associated diminished or altered state of consciousness. The definition of TBI has not been consistent and tends to vary according to specialties and circumstances. Often, the term brain injury is used synonymously with head injury, which may not be associated with neurologic deficits. The definition also has been problematic with variations in inclusion criteria.

TBI can be classified based on severity, mechanism (closed or penetrating head injury), or other features (e.g. occurring in a specific location or over a widespread area). Head injury usually refers to TBI, but is a broader category because it can involve damage to structures other than the brain, such as the scalp and skull. Brain trauma can be caused by a direct impact or by acceleration alone. In addition to the damage caused at the moment of injury, brain trauma causes secondary injury, a variety of events that take place in the minutes and days following the injury. These processes, which include alterations in cerebral blood flow and the pressure within the skull, contribute substantially to the damage from the initial injury. TBI can cause a host of physical, cognitive, emotional, and behavioral effects, and outcome can range from complete recovery to permanent disability or death.

In yet another embodiment, the invention may be applicable for treating chronic brain injuries. Chronic brain injuries are defined as conditions characterized by persistent brain damage or dysfunction as sequelae of cranial trauma. This disorder may result from diffuse axonal injury; intracranial hemorrhages; brain edema; and other conditions. Clinical features may include dementia; focal neurologic deficits; persistent vegetative state; akinetic mutism; or coma. Chronic brain injury is sometimes referred to as post-traumatic, chronic encephalopathy, post-concussive chronic encephalopathy, chronic traumatic encephalopathy, chronic post-traumatic encephalopathy, chronic post-concussive syndrome, chronic post-concussive encephalopathy, brain, chronic injury and post-concussive syndrome.

It should be further recognized that in certain embodiments, the invention also encompasses combinations, compositions, kits and methoxs for treating the above outlined disorders using of the combinations of ambroxol and glucocerebroside as described above, and optionally, with further additional therapeutic agent.

As noted above, the novel combination of the invention, either in a combined composition or in a combined treatment regimen, is shown herein as immunomodulatory combination, that significantly reduce the levels of inflammatory cytokines, elevates anti-inflammatory cytokines and modulates regulatory lymphocytes. Thus, in yet a further aspect, the invention further provides methods for the treatment, prophylaxis, amelioration, inhibition or delaying the onset of an immune-related disorder in a subject, by administering to said subject a therapeutically effective amount of at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof and at least one beta-glycolipid, specifically, ambroxol and glucocerebroside.

An “Immune-related disorder”, as used herein encompasses any condition that is associated with the immune system of a subject, either through activation or inhibition of the immune system, or that can be treated, prevented or diagnosed by targeting a certain component of the immune response in a subject, such as the adaptive or innate immune response. The immune-related disorder may be a chronic inflammatory condition, specifically, any one of an inflammatory disease, viral infections, an autoimmune disease, metabolic disorders and a proliferative disorder, specifically, cancer.

A subset of immune-mediated diseases specifically applicable in the present invention, is known as autoimmune diseases. As used herein autoimmune diseases arise from an inappropriate immune response of the body against substances and tissues normally present in the body. In other words, the immune system mistakes some part of the body as a pathogen and attacks its own cells. This may be restricted to certain organs (e.g. in autoimmune thyroiditis) or involve a particular tissue in different places (e.g. Goodpasture's disease which may affect the basement membrane in both the lung and the kidney). Autoimmune disease are categorized by Witebsky's postulates (first formulated by Ernst Witebsky and colleagues in 1957) and include (i) direct evidence from transfer of pathogenic antibody or pathogenic T cells, (ii) indirect evidence based on reproduction of the autoimmune disease in experimental animals and (iii) circumstantial evidence from clinical clues.

It should be further appreciated that in general, the combination of the present invention as well as the methods, and kits of the present invention may be used in preventing, treating, ameliorating or inhibiting any autoimmune disease such as for example, but not limited to, Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, systemic lupus erythematosus (SLE), multiple sclerosis (MS), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjogren's syndrome, allergic purpura, psoriasis, juvenile idiopathic arthritis, gout and pseudo gout mixed connective tissue disease, polymyositis, dermatomyositis, vasculitis, polyarteritis nodosa, polymyalgia rheumatica, Wegener's granulomatosis, Reiter's syndrome, Behget's syndrome, ankylosing spondylitis, pemphigus, bullous pemphigoid, dermatitis herpetiformis, inflammatory bowel disease, ulcerative colitis and Crohn's disease and fatty liver disease.

“Chronic inflammatory condition”, as used herein is reflected by an inflammatory response. As used herein the phrase “inflammatory response” refers to an immune response which results in either chronic or acute inflammation, typically occurring as a result of injurious stimuli including infection, burns, trauma, neoplasia, autoimmune signals and exposure to chemicals, heat or cold or any other harmful stimulus. An inflammatory response according to the present invention refers to a chronic inflammation.

Still further, in some embodiments, the immunomodulatory effect of the combination of the invention may be also applicable for treating proliferative disorders. As used herein to describe the present invention, “cancer”. “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for non-solid and solid tumors.

Malignancy, as contemplated in the present invention may be selected from the group consisting of carcinomas, melanomas, lymphomas and sarcomas. Malignancies that may find utility in the present invention can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including lung, liver, breast, colon, prostate GI tract, pancreas and Karposi). More particularly, the malignant disorder may be hepaotcellular carcinoma, colon cancer, melanoma, myeloma, acute or chronic leukemia. Non-limiting examples of cancers treatable by the combination according to the invention include hematopoietic malignancies such as all types of lympxhomas, leukemia, e.g. acute lympho ytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma. The combination of the invention may be also applicable for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

As used herein. “disease”. “disorder”. “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

As noted above, the invention provides methods for treating disorders as specified above. The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above. The treatment may be undertaken when a neuro-pathological condition initially develops, or may be a continuous administration, for example by administration more than once per day, every 1 day to 7 days, every 7 day to 15 days, every 15 day to 30 days, every month to two months, every two months to 6 months, or even more, to achieve the above-listed therapeutic effects.

The term “prophylaxis” refers to prevention or reduction the risk of occurrence of the biological or medical event, specifically, the occurrence or re occurrence of disorders associated with neurodegeneration, that is sought to be prevented in a tissue, a system, an animal or a human being, by a researcher, veterinarian, medical doctor or other clinician, and the term “prophylactically effective amount” is intended to mean that amount of a pharmaceutical composition that will achieve this goal. Thus, in particular embodiments, the methods of the invention are particularly effective in the prophylaxis, i.e., prevention of conditions associated with neurodegenerative disorders. Thus, subjects administered with said compositions are less likely to experience symptoms associated with said neurodegenerative disorders that are also less likely to re-occur in a subject who has already experienced them in the past.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the neurodegenerative disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a disorder associated with protein misfolding or protein aggregation, specifically, neurodegenerative disorders and their symptoms slowing their progress, further exacerbation or development, so as to appear later than in the absence of the treatment according to the invention.

As noted above, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”. “reduction” or “attenuation” as referred to herein, relate to the retardation, restraining or reduction of a process, specifically, a neurodegenerative disorder by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

As indicated above, the method of the invention involves the administration of a therapeutically effective amount of the combinations of the invention. The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the distribution profile within the body, a variety of pharmacological parameters such as half-life in the body, on undesired side effects, if any, on factors such as age and gender, etc.

More specifically, the combinations, compositions or kits comprising the beta-glycolipids, specifically, glucocerebroside and the trans-4-(2-Amino-3, 5-dibromobenzylamino) cyclohexanol hydrochloride, specifically, ambroxol provided by the invention, or any combination, mixture or cocktail thereof may be administered for prophylactic and/or therapeutic treatments. In therapeutic application, compositions are administered to a patient already affected by a neurodegenerative disease or specifically, a disorder associated with protein misfolding and protein aggregations, in an amount sufficient to cure or at least partially arrest the condition and its complications. An amount adequate to accomplish this is defined as a “therapeutically effective dose.” Amounts effective for this use will depend upon the severity of the condition, but generally range from about 0.01 to about 10.000 mg/kg, specifically, about 0.01 to about 1000, 500, 250, 100, 90, 80, 70, 60, 50, 40, 30, 20, 10, 9, 8, 7, 6, 5, 4, 3, 2, 1, 0.1 mg/kg. It should be noted that in certain embodiments, the effective amount of GC may be 1, 1.5, 2, 2.5, 3, 3.5, 4, 4.5, 5, 5.5, 6, 6.5, 7, 7.5, 8, 8.5, 9, 9.5, 10 mg/kg or more, specifically. 7.5 mg. In yet some further embodiments, for Ambroxol the effective amount may range between 0.000001 mg/kg to 1000 mg/kg, specifically, 300, 600, 900 mg/kg or more.

Single or multiple administrations on a daily, weekly or monthly schedule can be carried out with dose levels and pattern being selected by the treating physician. More specific embodiments relate to the use of typically 2-3 doses per week.

The present invention relates to the treatment of subjects, or patients, in need thereof. By “patient” or “subject in need” it is meant any organism who may be infected by the above-mentioned pathogens, and to whom the preventive and prophylactic kit/s, system/s and methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal.

More specifically, the combination/s, composition/s, kit/s and method/s of the invention are intended for preventing pathologic condition in mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral lavage and directly into the digestive tract of subjects in need thereof.

Still further, the combination/s, composition/s and kit/s of the invention and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the combination/s, composition/s and kit/s of the invention or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

It should be appreciated that the method of the invention are not limited to any rout of administration. Specifically, the combination/s, composition/s and kit/s may be administered either systemically, or locally, for example, topically. The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers, or dry powder dispersion devices.

The invention further encompasses the use of the combinations of the invention for treating any condition related to the disorders described above.

One embodiment provides a combination comprising 4-[(2-amino-3, 5-dibromophenyl)methylamino]cyclohexan-1-ol and at least one beta-glycolipid or any composition or kit comprising the same for use in the treatment of disorders involved in protein misfolding and protein aggregation.

Other embodiments provide the combination for use according to the invention, wherein said 4-[(2-amino-3, 5-dibromophenyl)methylamino]cyclohexan-1-ol or any pharmaceutically acceptable salt, solvate, esters, hydrate, stereoisomer or physiologically functional derivative thereof, may be ambroxol.

Still additional embodiments of the invention provide the combinations for use in accordance with the invention, wherein said beta-glycolipid is at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside, GM3 ganglioside, globoside or any derivative or combinations thereof.

In some specific embodiments, the combinations for use, in accordance with the invention may comprise ambroxol and glucocerebroside.

It should be appreciated that in yet some further embodiments, the combination for use according to the invention may further comprise at least one additional therapeutic drug. In more specific embodiments such drug is a drug having neuroprotective properties.

In still further embodiments, the combinations for use in accordance with the invention may be specifically suitable for treating disorders involved in protein misfolding and protein aggregation. In some specific embodiments, such disorders or conditions may be neurodegenerative disorders.

In yet more specific embodiments, the invention provides combinations as described above for use in disorders characterized by alpha-synuclein pathology. In more specific embodiments, such disorders may include but are not limited to PD, DLB MSA.

In still further specific embodiments, the invention provides the use of the combinations described herein before in the treatment of Parkinson disease or any conditions, symptoms, dementia or cognitive decline associated therewith. In yet some further specific embodiments, the invention provides the described combinations for use in the treatment of DLB. In yet another embodiment, the combinations of the invention are provided for use in the treatment of MSA.

In still further yet additional embodiments, the combinations of the invention may be provided for use in the treatment of disorders characterized by beta-amyloid protein aggregation, specifically, AD. In yet some further embodiments, the combinations of the invention may be provided for use in treating age-related cognitive decline, specifically. Mild Cognitive Impairment (MCI).

In a further aspect, the invention provides the use of an affective amount of a combination of at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof and at least one beta-glycolipid in the preparation of a pharmaceutical composition for the treatment of disorders involved in protein misfolding and protein aggregation.

According to some embodiments, the combinations used by the invention may comprise trans-4-(2-Amino-3,5-dibromobenzylamino) cyclohexanol hydrochloride and at least one beta-glycolipid.

According to some embodiments, an affective amount of the combination of the invention is used in the preparation of a pharmaceutical composition for the treatment of neurodegenerative disorders.

According to more specific embodiments, an affective amount of the combination of the invention may be used in the preparation of a pharmaceutical composition for the treatment of disorders involved in alpha-synuclein pathology.

In some specific embodiments, such disorders include any one of Parkinson disease, Dementia with Lewy Bodies (DLB) and multiple system atrophy (MSA).

According to yet further embodiment an affective amount of the combination of the invention may be used in the preparation of a pharmaceutical composition for the treatment of DLB.

According to an additional embodiment an affective amount of the combination of the invention may be used in the preparation of a pharmaceutical composition for the treatment of MSA.

According to some specific embodiments an affective amount of the combination of the invention may be used in the preparation of a pharmaceutical composition for the treatment of neurodegenerative disorders characterized by beta-amyloid protein aggregation. According to some further embodiments, an affective amount of the combination of the invention is used in the preparation of a pharmaceutical composition for the treatment of at least one of AD, ACD or MCI.

Still further, it should be appreciated that the invention provides the use of at least one beta-glycolipide, specifically, GC in the treatment of neurodegenerative disorders in subjects that are being treated with ambroxol, or that were previously treated with ambroxol.

In yet some further embodiments, the invention further provides the use of ambroxol in the treatment of neurodegenerative disorders in subjects that are being treated with or were previously treated with beta-glycolipides, specifically, GC.

It should be understood that the invention further encompasses at least one of GC, ambroxol or any combinations thereof for use in treating neurodegenerative disorders in subjects that are being treated or previously treated with at least one neuroprotective drug, specifically, any of the drugs disclosed by the invention.

As shown by Example 1, the novel combination of the invention exerts synergistic immunomodulatory effect. Thus, in yet another aspect, the present invention provides an affective amount of a combination of at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof and at least one beta-glycolipid for use in a method for modulating an immune response in a subject in need thereof, specifically, modulate the serum levels of at least one cytokine in a subject in need thereof.

In some embodiments, the combination of the invention may be used for down regulating, decreasing or reducing the serum levels of pro-inflammatory cytokines and/or up regulating, increasing elevating the serum levels of anti-inflammatory cytokines in a subject in need thereof.

The term “inflammatory cytokines” as used herein refers to signal molecules that are related to and indicative for inflammation response. The inflammatory indicative cytokines can be sub-grouped into two types: pro-inflammatory cytokines and anti-inflammatory cytokines. A pro-inflammatory cytokine, also known as inflammatory cytokine, is a signaling molecule excreted from immune cells like helper T cells (Th) and macrophages, and certain other cell types that promote inflammation. Pro-inflammatory cytokines include but are not limited to interleukin-1 (IL-1), IL-6, IL-12, and IL-18, tumor necrosis factor (TNF), interferon gamma (IFN-γ), and granulocyte-macrophage colony stimulating factor and play an important role in mediating the innate immune response. Inflammatory cytokines are predominately produced by and involved in the upregulation of inflammatory reactions. Anti-inflammatory cytokines are a series of immunoregulatory molecules that control the pro-inflammatory cytokine response. Cytokines act in concert with specific cytokine inhibitors and soluble cytokine receptors to regulate the human immune response. Their physiologic role in inflammation and pathologic role in systemic inflammatory states are increasingly recognized. Major anti-inflammatory cytokines include but are not limited to, interleukin (IL)-1 receptor antagonist, IL-4, IL-10, IL-11, and IL-13. It should be noted that IL-6 in some instances can act as an anti-inflammatory cytokine as well. Specific cytokine receptors for IL-1, tumor necrosis factor-alpha, and IL-18 also function as pro-inflammatory cytokine inhibitors.

In some embodiments, the combination of the present invention may be used for downregulating or reducing the serum levels of at least one of IL-1α, IFN-γ and IL-6 in a subject in need.

In certain embodiments, the combination of the present invention is used for upregulating, increasing or elevating the serum levels of IL-4 in a subject in need thereof.

Still further, in some embodiments, the combination of the present invention may be also used for modulating levels and distribution of T cells, specifically regulatory T cells and/or natural killer T cells.

It is to be understood that the terms “inhibition”, “moderation”, “reduction” or “attenuation” as referred to herein, relate to the retardation, restraining or reduction of pro-inflammatory cytokines by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or 100%.

With regards to the above, it is to be understood that, where provided, percentage values such as, for example, 10%, 50%. 120%, 500%, etc., are interchangeable with “fold change” values, i.e., 0.1, 0.5, 1.2, 5, etc., respectively.

It should be appreciated that the terms “increase”. “elevation”, “enhancement” or “enlargement” as referred to herein, relate to increase or upregulation in the levels of anti-inflammatory cytokines by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%. 120%, 500%, etc., are interchangeable with “fold change” values, i.e., 0.1, 0.5, 1.2, 5, etc., respectively.

Regulatory T cells (also known as Tregs) are a specialized subpopulation of T cells that act to suppress immune response, thereby maintaining homeostasis and self-tolerance. It has been shown that Tregs are able to inhibit T cell proliferation and cytokine production and play a critical role in preventing autoimmunity. Different subsets with various functions of Treg cells exist. Dysregulation in Treg cell frequency or functions may lead to the development of autoimmune disease. Regulatory T cells come in many forms with the most well-understood being those that express CD4. CD25. CD3 and FOXP3 (CD4+CD25+ regulatory T cells).

Therapeutical Treg modulation is considered to be a promising therapeutical approach to treat some selected disorders.

Natural killer T (NKT) cells are a heterogeneous group of T cells that share properties of both T cells and natural killer cells. NKT cells are a subset of T cells that co-express an af T-cell receptor, but also express a variety of molecular markers that are typically associated with NK cells, such as NK1.1.

In some embodiments, the combination of the present invention is used for downregulating, reducing or decreasing the levels of CD8+CD25+ in a subject in need thereof.

Still further, the combination of the present invention may be also used for alleviating immune-mediated liver injury. In a specific embodiment, the combination of the present invention may be used for modulating Aspartate transaminase (AST) and Alanine transaminase (ALT). In a more specific embodiment, the combination of the present invention is used for reducing the serum levels of at least one of AST and ALT or AST/ALT ratio in a subject in need.

Alanine transaminase (ALT) is a transaminase enzyme. ALT is found in plasma and in various body tissues, but is most common in the liver. It catalyzes the two parts of the alanine cycle.

Aspartate transaminase (AST) catalyzes the reversible transfer of an α-amino group between aspartate and glutamate and, as such, is an important enzyme in amino acid metabolism. AST is found in the liver, heart, skeletal muscle, kidneys, brain, and red blood cells.

Serum ALT level, serum AST (aspartate transaminase) level, and their ratio (AST/ALT ratio) are commonly measured clinically as biomarkers for liver health. Most causes of liver cell injury are associated with a greater increase in ALT than AST.

In yet another aspect, the present invention provides an affective amount of a combination of at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof and at least one beta-glycolipid for use in a method for preventing, treating, ameliorating or inhibiting immune-related disorders.

In a further aspect, the invention provides the use of an affective amount of a combination of at least one 4-[(2-amino-3, 5-dibromophenyl)methylamino]cyclohexan-1-ol, or any pharmaceutically acceptable salt, solvate, esters, hydrate, stereoisomer or physiologically functional derivative thereof and at least one beta-glycolipid in the preparation of a pharmaceutical composition for the treatment of immune-related disorders.

All scientific and technical terms used herein have meanings commonly used in the art unless otherwise specified. The definitions provided herein are to facilitate understanding of certain terms used frequently herein and are not meant to limit the scope of the present disclosure.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”. “an” and “the” include plural referents unless the content clearly dictates otherwise.

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B): in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A): in yet another embodiment, to at least one, optionally including more than one. A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. The terms “comprises”, “comprising”, “includes”, “including”. “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

In the claims, as well as in the specification above, all transitional phrases such as “comprising,” “including,” “carrying,” “having.” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”. “includes”. “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989,1992), and in Ausubel et al., Current Protocols in Molecular Biology, John Wiley and Sons, Baltimore. Md. (1988).

Standard organic synthesis protocols known in the art not specifically described herein are generally followed essentially as in Organic syntheses: Vol. 1-79, editors vary. J. Wiley. New York. (1941-2003); Gewert et al., Organic synthesis workbook, Wiley-VCH, Weinheim (2000); Smith & March. Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Vanderkerken K The 5T2MM murine model of multiple myeloma: maintenance and analysis [Methods Mol. Med. 113:191-205 (2005); Epstein J. The SCID-hu myeloma model. Methods Mol. Med. 113:183-90 (2005)].

Materials

Ambroxol hydrochloride which is bought as an OTC medication and was dissolved in distilled water to obtain Ambroxol solution.

B-glucosylceramide (GC) was purchased from Avanti Polar Lipids (Alabaster, Ala. USA) and dissolved in a mixture of 30% Cremophor EL (Sigma. Rehovot, Israel) and ethanol (1:1) in PBS.

Experimental Procedures

Pre-Clinical Trials

Animals

Male C57Bl/6 mice (12 weeks old) were obtained from Harlan Laboratories (Jerusalem, Israel) and maintained in the Animal Core of the Hadassah-Hebrew University Medical School. Mice were kept at 11-12 weeks of age and maintained in the Animal Core of the Hadassah-Hebrew University Medical School. All mice were administered standard laboratory chow and water ad libitum and kept in a 12-hour light/dark cycle. All experiments were performed in accordance with the guidelines of the Hebrew University-Hadassah Institutional Committee for Care and Use of Laboratory Animals (IACUC protocol number: MD-16-14986-3).

Experimental Groups

Four groups of mice (n=4) were studied. Mice in all groups were injected with Concanavalin A (Con A, 500 mg/mouse). Mice in control group A were treated with PBS. Mice in group B were orally administered 6 mg of GC, per mouse daily for 5 days prior to ConA injection. Mice in group C were orally administered 1.4 mg of Ambroxol per mouse daily for 5 days. Mice in group D were orally administered combination of 6 mg of GC and 1.4 mg of Ambroxol per mouse daily for 5 days.

Histological Examination

Livers of all the mice in all the experimental groups were cut into 4-5 μm thin slices, fixed in 10% formaldehyde solution, and kept at room temperature. Tissue blocks were embedded in paraffin. Sections were stained with hematoxylin-eosin (H&E) for morphological examination. Specimens were examined under a light microscope.

Liver Enzymes Determination

Serum aspartate aminotransferase (AST) and alanine aminotransferase (ALT) levels were determined with an automatic analyzer on all mice in all groups.

Assessment of the Effect of GC and Ambroxol on the Systemic Immune System

The immune modulatory effect determined by FACS analysis and serum cytokines.

Flow Cytometry on Isolated Splenocytes and Hepatic Lymphocytes

Splenocytes and hepatic lymphocytes were isolated as previously described [Trop S. et al. Hepatology 29:746-55 (1999); Falcone M, et al. J Immunol 172:5908-16 (2004)]. Briefly, approximately 1×10⁶ cells/mouse liver were recovered. Flow cytometry was performed on splenocytes and hepatic lymphocytes, which were resuspended in 1 mL of FACS buffer (PBS+1% BSA+0.1% sodium azide). Cells were stained with the diluted anti-LAP antibody (50 μL/sample). FITC-conjugated anti-CD4/CD8 (0.5 μL per sample), PE-conjugated anti-CD25/NK1.1 FITC-conjugated anti-CD3 (1 μL per sample), and PerCP-conjugated anti-CD45 (2 μL per sample). All stains were performed after blocking the Fc receptor with anti-mouse CD16/CD32 (BD Fc Block). Flow cytometry was performed using a LSR-IIflow cytometer and FCS express software.

Cytokine Measurement

Cytokine assessment was performed by MILLIPLEX® Analytes (EMD Millipore Corporation, Missouri 63304 U.S.A.) based on the Luminex xMAP® technology for performing immunoassays on the surface of fluorescent-coded magnetic beads MagPlex®-C microspheres. Acquiring and analyzing the data were performed using the Luminex analyzer (MAGPIX®) software. Cytokines assessment was measured by mean fluorescence intensity (MFI).

Statistical Analysis

All analysis was performed using Excel 2003 (Microsoft, Redmond, Wash., United States). The variables were expressed as the mean±SD. The comparison of two independent groups was performed using Student's t-test. All tests applied were two-tailed. A p value of 0.05 or less was considered statistically significant.

Clinical Trials

Subjects

Subjects participating in the clinical trial are selected in accordance with the following inclusion and exclusion criteria:

Inclusion Criteria

1. Signed informed consent.

2. Age 40-75 years.

3. Carrier of a known GBA mutation.

4. At least one of the following PD prodromal signs/symptoms:
Impaired sense of smell demonstrated in objective smell test.
UPDRS III excluding action tremor >3.
Abnormal substantia nigra (SN) ultra-sound hyperechogenicity (>0.2).
Constipation based on validated scale
Thinning of the retina measured by OCT
REM sleep disorder (validated)

Exclusion Criteria

1. Established diagnosis of Gaucher disease

2. Established diagnosis of Parkinson's disease. Lewy body dementia (DLBD) or other neurodegenerative disease at the time of screening.

3. Treatment with MAO-B inhibitors or any symptomatic, anti-Parkinsonian, therapy at the time of screening.

4. Significant cognitive impairments (MoCA<26 for participants with 12 years or more of education or MoCA<25 for participants with less than 12 years of education) at the time of screening.

5. History of psychosis.

6. History of exposure to dopamine receptor blocking agents, lithium or anti-epileptic drugs on the previous year.

7. Clinical depression.

8. Pregnancy or lactation, or female subject at childbearing age who is unwilling to use contraceptive measures.

9. Use of another experimental treatment.

Procedures for Clinical Trial

Follow-Up of Subjects

Screening (week −2±1 week):
Informed consent.
Review of inclusion and exclusion criteria.
Review of medication, medical conditions, relevant family history, allergies and habits.
General physical exam.
Vital signs (pulse and BP sitting and standing)
ECG
Video-recorded UPDRS
Smell test
MoCA
Blood test—CBC, biochemistry (including renal and liver). LysoGb
Brain-stem US.
OCT.
The Patient Global Impression of Improvement (PGI-I)
Parkinson's disease questionnaire (PDQ-39)
Epworth Sleepiness Scale
Beck Depression Inventory
Frontal assessment battery (FAB)
Addenbrcxoke's Cognitive Examination
Questionnaire for Impulsive-Compulsive Disorders in Parkinson's (QUIP-RS)

Visit 1 (time 0):
Review of inclusion and exclusion criteria.
Review of medication, medical conditions, relevant family history, allergies and habits.
Vital signs (pulse and BP sitting and standing)
Review of blood test results.
Video-recorded UPDRS
Neurotrax
Blood test—CBC, biochemistry (including renal and liver)
Brain-stem US.
OCT.
Timed up-and-go test.
Purdue pegboard.
Randomization and receiving the study drug—placebo/300 mg Ambroxol+7.5 mg GC.

Visit 2 (month 1±1 week):
Review of inclusion and exclusion criteria.
Review of medications, medical conditions.
General physical exam.
Vital signs (pulse and BP sitting and standing).
Blood test—CBC, biochemistry (including renal and liver), Ambroxol level, LysoGb1.
ECG
Review of AE's (Adverse Events)
Increase dose to 600 mg Ambroxol+7.5 mg GC.

Visits 3 (month 2±1 week):
Review of inclusion and exclusion criteria.
Review of medication, medical conditions.
General physical exam.
Vital signs (pulse and BP sitting and standing).
Blood test—CBC, biochemistry (including renal and liver). Ambroxol level. LysoGb1.
Review of AE's
Increase dose to 900 mg Ambroxol+7.5 mg GC.

Visits 4 (Month 3±1 Week):
Review of inclusion and exclusion criteria.
Review of medication, medical conditions.
General physical exam.
Vital signs (pulse and BP sitting and standing).
Blood test—CBC, biochemistry (including renal and liver), Ambroxol level, LysoGb1.
Review of AE's

Visits 5 (Month 6±2 Weeks):
Review of inclusion and exclusion criteria.
Review of medication, medical conditions.
General physical exam.
Vital signs (pulse and BP sitting and standing).
Video-recorded UPDRS part III
Blood test—CBC, biochemistry (including renal and liver), Ambroxol level, LysoGb1.
Review of AE's

Visits 6 (Month 12±2 Weeks):
Review of inclusion and exclusion criteria.
Review of medication, medical conditions.
General physical exam.
Vital signs (pulse and BP sitting and standing).
Video-recorded UPDRS part III
Blood test—CBC, biochemistry (including renal and liver). Ambroxol level. LysoGb1.
Review of AE's

Final Visit (Month 18±2 Weeks):
Review of medication, medical conditions.
General physical exam.
Vital signs (pulse and BP sitting and standing).
Video-recorded UPDRS Total
Timed up-and-go test
Purdue pegboard
MoCA
Smell test
OCT
Mid-brain OCT
Review of AE's
Blood test—CBC, biochemistry (including renal and liver)

Randomization:

Subjects are randomized based on their sex and severity of GBA mutation (N370S or R496H versus non N370S non R496H). After completing screening, patient are sorted into one of four groups: male with mild GBA mutations, male with severe GBA mutation, female with mild GBA mutation or female with severe GBA mutation. Each of the indicated group is equally distributed between the four experimental groups (ABX+GC, GC alone, ABX alone and the placebo arm).

Trial Blindness:

The investigators are blinded to the randomized agent. Safety endpoint at three months are performed by external committee and the investigator blindness is not broken at the point.

Genetics:

All candidates are GBA carriers with a known mutation.

Recruitment:

Subject are recruited at the Gauche clinic at Shaare-Zedek medical center. Recruitment pool includes only subject with known GBA mutation.

### Example 1

An Immunomodulatory Effect for Ambroxol in Combination with Aglucosylceramide (GC) in Mice Model of an Immune-Mediated Disorder

Neurodegenerative disorders such as PD, were shown as exhibiting intimate connection with neuro-inflammatory processes that involve activation and production of pro-inflammatory cytokines. Current methods for treatment of immune-mediated and autoimmune disorders usually involve use of immunosuppressive agents. These drugs are associated with side effects, many of which are related to a direct suppressive effect on various arms of the immune system. Thus, in an attempt to develop immunomodulatory agents which will not exert immunosuppressive effects and therefore may be safely applicable in the treatment and prevention of neurodegenerative disorders, the inventors examined the immunomodulatory effect of novel combined administration of Ambroxol with GC. Immune-mediated hepatitis induced by administration of ConA was used in the present invention as a model for inflammatory disorders.

Several immunomodulatory aspects that may be also relevant to neuronal inflammation examined in this model, specifically, the effect on different subsets of lymphocytes, expression of pro-inflammatory and anti-inflammatory cytokines, the effect on liver enzymes (ALT and AST) and apoptosis, indicated a clear synergistic effect of the novel combination of the invention.

More specifically. FIGS. 1A-1C show a synergistic effect of oral administration of Ambroxol and GC on different subsets of lymphocytes. FIG. 1A shows a decrease in hepatic CD8⁺CD25+ lymphocytes only in mice treated with the combination of Ambroxol and GC in group D compared to the untreated mice in group A (6.09% vs. 11.25% for groups D and A, respectively). FIG. 1B shows a clear synergistic effect of the combination of Ambroxol and GC on the CD4/CD8 lymphocyte ratio. The CD4\CD8 ratio in the spleen increased in the combination treated group compared to the other groups. No significant effect was noted for the CD4/CD8 ratio in the liver.

As shown by FIG. 1C, the intrasplenic/intrahepatic ratio of the CD4/CD8 ratios was markedly increased only in the combination treated group D compared to the other groups, suggesting the sequestration of CD8+ positive cells in the liver.

The data of the present invention support redistribution of different subsets of regulatory lymphocytes (for example, CD8+CD25+ lymphocytes that are known to exert immunosuppressive functions in various organs), as part of the overall regulatory immunomodulation exerted by treatment with the novel combination of the invention. The increase in FoxP3 cells further support promotion of regulatory lymphocytes in the treated groups. Thus, the novel combination of the invention exerts a synergistic effect on specific regulatory lymphocytes.

The inventors next examined the effect of the novel combination of the invention on secretion of pro-inflammatory and anti-inflammatory cytokines as an additional parameter establishing the immunomodulatory effect of this combination. FIGS. 2A-2D show a shift of pro- and anti-inflammatory cytokines associated with oral administration of Ambroxol with GC. More specifically, FIG. 2A shows a statistically significant decrease in the level of serum IL-1a, a pro-inflammatory cytokine, in treated groups (206.5 vs. 48 vs. 131 vs. 42 pg/ml for groups A, B. C and D respectively, P=0.03 for group D vs. A, P=0.44 for group D vs. B and P=0.01 for group D vs. C). FIG. 2B shows a statistically significant increase in the serum level of the anti-inflammatory cytokine IL-4 in group D as compared to group B and C (P=0.02 for group D vs. B and P=0.01 for group D vs. C). FIG. 2C shows a statistically significant decrease in the pro-inflammatory cytokine IFN-γ in groups B and D compared to group A (36 and 19 vs. 643 pg/ml, respectively, P=0.0004 and P=0.00009 for group B and D compared to group A). Similarly, there was a statistically significant decrease in group D compared to C (P=0.01). Group C did not show any decrease in IFN-γ. FIG. 2D shows a significant decrease of serum IL-6 in treated groups (11768 vs. 984 vs. 9504 vs. 567 pg/ml for groups A, B, C and D respectively, P=0.00002 for group D vs. A, P=0.29 for group D vs. B and P=0.0001 for group D vs. C). However, the decrease in group C compared to group A did not reach statistical significance (P=0.14). These results clearly establish the immunomodulatory effect of the combination of the invention in reducing the levels of pro-inflammatory cytokines and increasing the serum levels of anti-inflammatory cytokines.

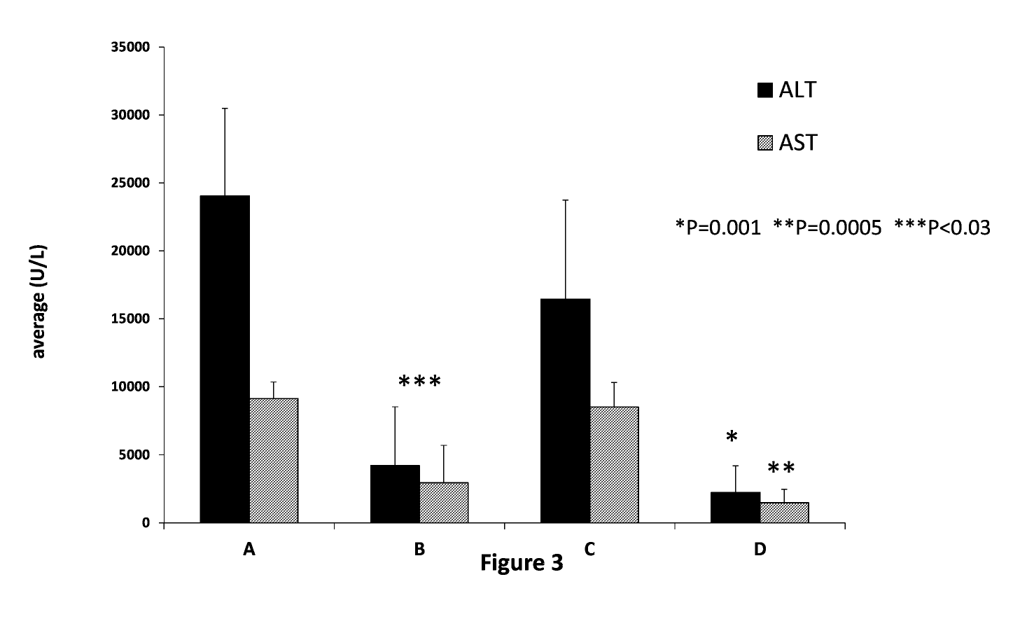

As a further parameter for inflammatory tissue damage, the inventors next examined the effect of the combination of the invention on liver enzymes. FIG. 3 shows the effect of oral administration of the either Ambroxol, GC, or a combination of Ambroxol and GC on immune-mediated liver injury as measured by the effect on liver enzymes (ALT and AST). The oral administration of combination treatment of Ambroxol and GC was associated with significant alleviation of liver injury compared with the untreated controls in group A (24040 vs. 4198 vs. 16455 vs. 2235 IU for ALT levels: and 9140 vs. 2939 vs. 8516 vs. 1470 IU for AST levels, for groups A. B, C, and D, respectively. P<0.05 for groups B and D vs. group A, P<0.004 for group D vs. C). Similarly, there was a significant decrease in the liver enzymes in group B vs. group A (P=0.004 and 0.0002 for ALT and AST respectively). Notably, the decrease in liver enzymes were more profound in group D compared to group B, however the difference did not reach statistical significance (P>0.1).

Still further, to assess the effect of the combination of the invention on tissue architecture and apoptosis, the inventors next examined the effect of this combination on inflammation induced apoptosis. FIG. 4 shows representative sections of liver biopsies performed at the end of treatment period. Alleviation of liver apoptosis and improved hepatocyte architecture were noted in mice in all treated groups compared to the untreated controls, thus establishing the protective effect of the combination of the invention.

In summary, co-administration of Ambroxol with GC exerted a synergistic immuno-protective effect in a model of immune-mediated disorder. Bearing in mind the high safety profile of both agents, these results establish the feasibility of using the combination of the invention as a novel immunomodulatory non-immunosuppressive therapeutic platform.

### Example 2

MPTP Mouse Model of Parkinson's Disease (PD)

For evaluating the neuroprotective effect of the combinations of the invention on alpha-synucleic disorders, the MPTP animal model was used (Bove J et al., Neurotoxin-based models of Parkinson's disease. Neuroscience 211 (2012) 51-76). More specifically, twenty male mice 8 weeks of age weighted et least 22 gram are subcutaneously injected with 1-methyl-4-phenyl-4-propionpiperidine (MPPP) at the schedule of total of four injections at a dose of 20 mg/kg with 2 hours intervals between injections. This regimen leads to 90% striatal dopamine depletion and about 70% loss of dopaminergic neurons characteristic for PD for 7 days. Ten mice are co-administered with the combination of ambroxol and GC 1 mg/kg), while the rest of the mice serve as a control. The protective effect of the combination of the invention on the neurodegeneration induced by MPTP is monitored and evaluated by the following parameters: 1—tail climbing test; 2—motor impairment tests: by grid coordination test (by measuring the forepaw stride length during walking, and the forepaw distance, wall time, and forepaw faults in the grid). The extent of neuronal death is assessed by histological analysis of mice brains in each group of mice. The neurological impairment is able to undergo a total or partial reversal by the rescue combination of ambroxol and GC in the study group mice.

### Example 3

Ambroxol and Glucocerebroside Combinations for the Treatment and Prevention of Parkinson Disease (PD) in Human Patients Having an Increased Risk for Developing PD

To evaluate the neuroprotective and prophylactic effect of the Ambroxol and glucocerebroside combination of the invention on human subjects, an eighteen months randomized, double-blind, placebo-controlled trial is conducted in carriers of glucocerebrosidase mutation at increased risk for developing Parkinson's disease, subjected to combined treatment with Glcuocerebroside and Ambroxol.

More specifically, since the risk of developing PD is significantly higher among male and among carriers of severe GBA mutations, subjects participating in the trial are randomized based on their sex and severity of GBA mutation (N370S or R496H versus non N370S non R496H) as indicated in the trial procedure herein before. The 100 subjects (male and female aged between 40-75) participating in the trial are divided into the following four experimental groups:

TABLE 1

| Group | Ambroxol | GC 7.5 mg daily |
|---|---|---|
| A | − | − |
| B | + | + |
| C | + | − |
| D | − | + |

The subjects are followed during the trial period at the beginning of the trail (time 0), after a month, two months, three, six, twelve and eighteen months (time 1, 2, 3, 6, 12 and 18, respectively). AEs is collected at each visit, in addition to ECG, blood count and biochemistry tests performed at each visit. Safety of the treatment (primary endpoint) is examined by an external safety board at 3 months and after 12 months. During the first six visits (time 0, 1. 2, 3, 6 and 12 months) the subjects are monitored as indicated in the experimental procedures herein above.

At the end of the trial (Secondary End Points at 18 months) the following parameters are evaluated in each subject: (1) The mean change in the motor score (part III) of the UPDRS score; (2) Mean change in total UPDRS score (I-III); (3) MoCA score; (4) Timed up-and-go test; (5) Purdue pegboard; (6) Neurotrax; (7) The Patient Global Impression of Improvement (PGI-I); (8) Parkinson's disease questionnaire (PDQ-39); (9) Epworth Sleepiness Scale: (10) Beck Depression Inventory; (11) Frontal assessment battery (FAB); (12) Addenbrooke's Cognitive Examination; and (13) Questionnaire for Impulsive-Compulsive Disorders in Parkinson's (QUIP-RS).

The subjects are further evaluated for the following exploratory End Points (at week 72): (1) Smell test between weeks 0 and 72; (2) Substantia nigra (SN) ultra-sound hyperechogenicity (>0.2); (3) Thinning of the retina measured by OCT; (4) Lyso Gb1; (5) Color discrimination test; and (6) Orthostatic hypotension.

The treatment is administered as indicated above and the improvement in neurological and/or cognitive symptoms and indications as reflected by the examined parameters is being monitored.

1. A combination comprising at least one 4-[(2-amino-3, 5-dibromophenyl)methylamino]cyclohexan-1-ol or any pharmaceutically acceptable salt, solvate, esters, hydrate, stereoisomer or physiologically functional derivative thereof and at least one beta-glycolipid or any derivatives or analogues thereof, or a composition comprising said combination.

2. The combination according to claim 1, wherein
(a) said 4-[(2-amino-3, 5-dibromophenyl)methylamino]cyclohexan-1-ol is trans-4-(2-Amino-3,5-dibromobenzylamino) cyclohexanol hydrochloride (ambroxol); and/or
(b) said beta-glycolipid is at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside, GM3 ganglioside, globoside or any derivative or combinations thereof.

3. (canceled)

4. The combination according to claim 1, wherein said combination comprises trans-4-(2-Amino-3,5-dibromobenzylamino) cyclohexanol hydrochloride and glucocerebroside, said combination optionally further comprising at least one additional therapeutic drug.

5. (canceled)

6. The combination according to claim 1, comprised in a pharmaceutical composition, wherein said composition comprising the combination of the at least one 4-[(2-amino-3, 5-dibromophenyl) methylamino]cyclohexan-1-ol or any pharmaceutically acceptable salt, solvate, esters, hydrate, stereoisomer or physiologically functional derivative thereof and the at least one beta-glycolipid or any derivatives or analogues thereof, said composition optionally further comprises at least one of pharmaceutically acceptable carrier/s, excipient/s, additive/s diluent/s and adjuvant/s, wherein said combination is in an effective amount for the treatment, prophylaxis, amelioration, inhibition or delaying the onset of a disorder involved in protein misfolding and/or protein aggregation or of any early signs or symptoms associated therewith.

7.-8. (canceled)

9. The pharmaceutical composition according to claim 6, wherein said disorder is a neurodegenerative disorder.

10. The pharmaceutical composition according to claim 9, wherein said neurodegenerative disorder is a disorder characterized by alpha synuclein pathology wherein said aloha-synuclein pathology is at least one of Parkinson disease (PD), Dementia with Lewy Bodies (DLB) and multiple system atrophy (MSA).

11. (canceled)

12. The pharmaceutical composition according to claim 10, for use in the treatment, prophylaxis, amelioration, inhibition or delaying the onset of at least one of (a) PD and/or any dementia, cognitive decline, early signs or symptoms associated therewith; (b) DLB, and/or any dementia, cognitive decline, early signs or symptoms associated therewith; and (c) MSA and/or any dementia, cognitive decline, early signs or symptoms associated therewith.

13.-14. (canceled)

15. The pharmaceutical composition according to claim 9, wherein said neurodegenerative disorder is a disorder characterized by beta-amyloid protein aggregation, wherein said beta-amyloid protein aggregation disorder is at least one of Alzheimer's disease (AD) and age-associated cognitive decline (ACD).

16. (canceled)

17. A kit comprising:
(i) at least one 4-[(2-amino-3, 5-dibromophenyl) methylamino]cyclohexan-1-ol or any pharmaceutically acceptable salt, solvate, esters, hydrate, stereoisomer or physiologically functional derivative thereof, optionally, in a first dosage form; and
(ii) at least one beta-glycolipid or any derivatives or analogues thereof, optionally, in a second dosage form.

18. The kit according to claim 17, wherein:
(a) said 4-[(2-amino-3, 5-dibromophenyl)methylamino]cyclohexan-1-ol is trans-4-(2-Amino-3,5-dibromobenzylamino) cyclohexanol hydrochloride (ambroxol); and/or
(b) said beta-glycolipid is at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside, GM3 ganglioside, globoside or any derivative or combinations thereof.

19. (canceled)

20. The kit according to claim 17, comprising ambroxol and glucocerebroside, wherein said kit optionally further comprises at least one additional therapeutic drug.

21.-27. (canceled)

28. A method for the treatment, prophylaxis, amelioration, inhibition or delaying the onset of a disorder involved in protein misfolding and/or protein aggregation, said method comprising the step of administering to a subject in need thereof an effective amount of at least one 4-[(2-amino-3, 5-dibromophenyl)methylamino]cyclohexan-1-ol or any pharmaceutically acceptable salt, solvate, esters, hydrate, stereoisomer or physiologically functional derivative thereof and at least one beta-glycolipid or any derivatives or analogues thereof or of any combinations thereof or any composition or kit comprising the same.

29. The method according to claim 28, wherein:
(a) said 4-[(2-amino-3, 5-dibromophenyl)methylamino]cyclohexan-1-ol is ambroxol; and/or
(b) said beta-glycolipid is at least one of glucocerebroside, glucosylceramide, glucosylsphingosine, lactosylceramide, glycosphingolipid, monosaccharide ceramide, galatosylceremide, gal-gal-glucosyl-ceramide, GM2 ganglioside, GM3 ganglioside, globoside or any derivative or combinations thereof.

30. (canceled)

31. The method according to claim 28, wherein said method comprises the administration of ambroxol and glucocerebroside or any combination thereof or any composition comprising the same.

32. (canceled)

33. The method according to claim 28, wherein said disorder is a neurodegenerative disorder.

34. The method according to claim 33, wherein said neurodegenerative disorder is a disorder characterized by alpha-synuclein pathology, and wherein said alpha-synuclein pathology is at least one of PD, DLB and MSA, optionally, said alpha synuclein pathology is PD and/or any conditions, dementia, cognitive decline, early signs or symptoms associated therewith.

35.-36. (canceled)

37. The method according to claim 28, for prevention of PD in a subject in need thereof, wherein said subject is at least one of a subject at risk for developing PD and a subject that displays early singes or symptoms associated with PD, and wherein said subject at risk for developing PD is a subject that carry at least one mutation in at least one gene encoding glucocerebrosidase (GBA) or any protein associated therewith.

38. (canceled)

39. The method according to claim 34, wherein said method results in improvement in at least one of: the mean change in the motor score (part III) of the unified Parkinson's disease rating scale (UPDRS score); Mean change in total UPDRS score (I-III); Montreal Cognitive Assessment (MoCA) score; Timed up-and-go test; Purdue pegboard; Neurotrax; The Patient Global Impression of Improvement (PGI-I); Parkinson's disease questionnaire (PDQ-39); Epworth Sleepiness Scale; Beck Depression Inventory; Frontal assessment battery (FAB); Addenbrooke's Cognitive Examination; Questionnaire for Impulsive-Compulsive Disorders in Parkinson's (QUIP-RS), Smell test, Substantia nigra (SN) ultra-sound hyperechogenicity (>0.2); Thinning of the retina measured by OCT; Lyso Gb1; Color discrimination test; and Orthostatic hypotension.

40. The method according to claim 34, wherein said alpha synuclein pathology is DLB and/or any conditions, dementia, cognitive decline, early signs or symptoms associated therewith, and wherein said alpha synuclein pathology is MSA and/or any conditions, dementia, cognitive decline, early signs or symptoms associated therewith.

41. (canceled)

42. The method according to claim 33, wherein said neurodegenerative disorder is a disorder characterized by beta-amyloid protein aggregation, and wherein said beta-amyloid protein aggregation disorder is AD or any age-related cognitive decline.

43.-53. (canceled)
